(19) United States
(12) Patent Application Publication
(10) Pub. No.: US 2014/0330288 A1
(43) Pub. Date: Nov. 6, 2014

(54) Articulating Arm for a Robotic Surgical Instrument System

(71) Applicant: Precision Automation and Robotics India Ltd., District Satara (IN)

(72) Inventors: Ranjit Date, Pune (IN); Jaydeep Date, Pune (IN); Mihir Desai, La Canada, CA (US)

(21) Appl. No.: 14/332,894

(22) Filed: Jul. 16, 2014

Related U.S. Application Data
(63) Continuation-in-part of application No. 13/069,067, filed on Mar. 22, 2011, now abandoned.
(60) Provisional application No. 61/282,740, filed on Mar. 25, 2010.

(51) Int. Cl. A61B 19/00

(52) U.S. Cl. CPC A61B 19/2203; A61B 2019/2234; B25J 9/0018
USPC 606/130

(57) ABSTRACT

An articulating arm for a robotic surgical instrument system includes arm elements, an elongate support element, end effectors and a wrist joint. The arm elements are joined by a first pitch joint. The elongate support element is connected at one end thereof to a first arm element by a second pitch joint and other end of the elongate support element is connected to a support structure and is functionally coupled to a control. The elongate support element, arm elements, pitch joints and end effectors are linearly inserted via an aperture of a port into an operation site and once inserted are articulated and moved to perform a single port procedure with at least seven axes of movement. The robotic surgical instrument system includes a pair of articulating arms, a control and a resilient access port.

### CROSS REFERENCE TO RELATED APPLICATIONS

This application is a continuation-in-part (CIP) of U.S. patent application Ser. No. 13/069,067, filed Mar. 22, 2011, now under examination claiming priority from U.S. patent application Ser. No. 61/282,740 dated Mar. 25, 2010, and the disclosure of which is incorporated by reference herein.

### FIELD OF THE DISCLOSURE

The present disclosure relates to the field of robotic surgical instrument systems.

### BACKGROUND

Surgery, typically involves an invasive procedure that requires multiple, large incisions and stitches, involves longer healing time due to the multiple and larger incisions that require more time to heal, risk of infection, and requires a patient to be under anesthesia for a longer period of time. Laparoscopic surgery, also referred to as minimally invasive surgery, is a boon that solves most of the aforementioned problems, besides being cosmetically appealing to a patient.

In case of laparoscopic surgery, an incision is made in a patient's abdomen and the incision may be retracted using a retractor. An access device is attached to the retractor. The access device has a number of access ports each with an instrument seal to effect a seal around a separate instrument extended through the device. Each instrument seal is separate from the other instrument seals and is spaced apart from the other instrument seals. The instrument seals may be used with various instruments and/or camera/scopes.

Robot assisted laparoscopic surgeries are performed with limited physical contact between a surgeon and a patient. The surgeon is remote from the patient, working a few feet from the operating table while seated at a computer console with a three-dimensional view of the operating field.

### Objects

A main drawback associated with robotic systems known in the art is the need for a plurality of incisions in a patient's body and accordingly a plurality of access ports for insertion of surgical arms of the robotic systems. One such robotic system disclosed in the Granted U.S. Pat. No. 6,843,793, wherein such robotic system requires three separate incisions, two for the surgical instruments and a centrally disposed incision for the viewing endoscope. Further, it is observed that the arm of U.S. Pat. No. 6,843,793 requires many movements outside patient's body above the access port mounted on an incision(s) formed on the patient's body. These movements at times are required to be stopped, that is, the operation needs to be stopped, for obvious reasons such as the surgeon might need to change the drape and again start the operation. This is certainly undesirable. Still further, the arm of U.S. Pat. No. 6,843,793 at the most is provided with seven degrees of freedom that does limit the maneuverability of the arm.

The surgical arms that can be inserted into an abdominal cavity via a single access port for performing surgical procedures are also known in the prior art. However, the conventionally known surgical arms provide movement about very few axes of movement and accordingly, fail to provide maneuverability, proper approach to an operative space, proper movement and triangulation, and accordingly, the surgeon fails to perform complex procedures while operating through the miniaturized single port. For example, the US Published Patent Application US2005096502 (hereinafter referred to as '502 US Published patent application) and US Published Patent Application US 20110213384 (hereinafter referred to as '384 US Published patent application) discloses a “Robotic surgical device” that includes an elongated body for insertion into a patient's body through a small incision, the elongated body is required to be precisely configured for introducing surgical arms. In one variation, the elongated body houses a plurality of robotic arms that are introduced via the precisely configured passages configured in the elongate body. However, in case of the robotic arms of the '502 US Published patent application and the '384 US Published patent application have to pass through the elongate body and displacement in this case is very limited because of inherent constructional configuration of the arm and its passage through the elongate body and the ability of the arms to perform gripping and other actions for performing the surgical procedures is very limited and the surgical arms can only approach very small regions, organs and tissues for performing small scale procedures only. Further, the robotic arms of the '502 US Published patent application fail to provide a feel of using multi-port robotic systems to which the surgeon have been used to in the past and the surgeon needs to be trained for using the robotic surgical device of the '502 US Published patent application. As the surgeons have been trained and using multi-port robotic systems and there is already a level of training and experience available for using surgical arms for multiport surgery. The conventionally known surgical arms used with single port robotic surgical system result in a completely new way of working, hence need for new training and skills requirement is felt. The conventional surgical arms used with the single port surgical system do not provide a feel of multi-port surgical system and the surgeons are required to be trained to be adapted to the single port surgical systems. The surgical arms that can be inserted inside operation site through single port are known in the prior art. However, such surgical arms has a construction that prohibits the surgical arms from remaining in close proximity thereby preventing such arms to pass through a single port and hence accessing an organ inside an operation site through the single port becomes in-convenient. Further, the US Published Patent Application US20110172648 (hereinafter referred to as '648 US Published patent application) discloses a tool for minimally invasive surgery and method for using the same, however, the surgical arms disclosed herein have limitations of configuration and movements, with such limitation in configuration and movements of the surgical arms, the surgical arms fails to achieve movement along seven axes of movement of the arm and also fails to approach and cater to a wide range of sizes of organs. The US Published Patent Application US20070287884 discloses bundled insertion of surgical arms. In such case the arms can approach the organs only in smaller envelope and it is not possible to simultaneously approach larger organs from both sides. Further, with such bundled insertion of surgical arms, the arms approach without triangulation of forces and the range of operative procedures that the surgical arms passing through the single port can perform is reduced. Further, due to above mentioned limitations of the conventional surgical arms used by surgeon, the surgeon fails to maintain ergonomic posture thereby resulting in surgeon's fatigue and increasing chances of error due to fatigue. More specifically, it is highly challenging for the surgeon to manually move the instruments held at the distal end of the surgical arms that are in close proximity to each other and high level of skill is required at the surgeon's end. Further, the conventionally known surgical arms used with single port surgery systems have less than 7 degrees of freedom. The conventionally known surgical arms that are introduced inside the operation site through single port configuration requires movement of the elements of the arms, triangulation of forces for insertion and approach, each requirement limits the movement of the surgical arm about an axis of movement and hence the surgical arm can only achieves movement along less than seven axis of movement and accordingly maneuverability of fingers disposed at distal end of the arm is greatly reduced and the movement of the arms is limited. Further, the conventionally known surgical arms provide roll movement at proximal end thereof and this roll movement cannot be used in triangulated position. Further, the conventionally known surgical arms require lot of movement outside a patient's body above an access port mounted on an incision formed on the patient's body as conventional arms fail to provide better leverage and the proximal ends of the arm is required to be moved by larger distances to achieve even small movements at the distal end of the surgical arm, thereby creating an uncomfortable environment for people working in close proximity (such as nurses, orderlies and surgical attendants) to the patient's body. Further, the surgical arms of the conventional robotic surgical system, that are introduced inside the operation site in a straight configuration and have an angle of approach in the range of 5 degrees to 10 degrees, require significantly more force to achieve a horizontal pull force at the tip of the fingers configured at the distal end of the surgical arm. Further, the surgical arms of the conventional robotic surgical system can approach and perform operation at regions, organs, tissues and objects of a limited, pre-determined size range inside said operation site.

There is felt a need to overcome this drawback and provide arms for a robotic surgical instrument system that requires only one access port and a single incision in a patient's body. There is a need of surgical arms for a robotic surgical system that can adjust according to size of regions, organs, tissues and objects to be approached inside the operation site and can approach and perform operation at regions, organs, tissues and objects having a wide size range. There is a need for arm for a robotic surgical instrument system that can provide movement along at least seven axes of movement of the arms. Further, there is a need for an arm that has such a configuration that enables the arms to be inserted inside an operation site and operate in close proximity, such feature of the arms combined with provision for movement along at least seven axes of movement of the arms enables the arms to conveniently approach and access the organ inside the operation site and ensure that the arms have access over a large work envelop. Further, there is a need for arms that have such a configuration that enables the arms to enter separately through a single port and simultaneously approach an organ inside the operation site from opposite sides. Further, there is a need for arms that can be independently inserted inside an operation site through apertures configured on a single access port mounted on an incision on a patient's body and achieve triangulation. Further, there is a need for arms that have such configuration that the roll, pitch and finger movement are provided at distal end, thereby enabling control of the arm from proximal end. There is felt a need for arms for use with a single port system that has an operation feel like being used for a multi-port system that surgeons are now used to. There is felt a need for arms that requires significantly reduced force to achieve the required horizontal pull force at the tip of the fingers configured at the distal end of the surgical arm.

Still further, there is a need for arms that achieves movement along at least seven axes of movement for enabling a surgeon to perform complex procedures while operating through a miniaturized single port and still maintaining proper posture and the method of achieving triangulation within the operative space. Furthermore, there is a need for arms that operate in conjunction with joysticks of a robotic surgical instrument system to minimize fatigue of the surgeon by ensuring ergonomic posture of the surgeon.

Some of the objects of the present disclosure, which at least one embodiment herein satisfies are as follows:

It is an object of the present disclosure to provide a robotic surgical instrument system having a plurality of arms that facilitates minimal invasive surgery and that are versatile to adjust according to size of regions, organs, tissues and objects to be approached inside the operation site.

Another object of the present disclosure is to provide a robotic surgical instrument system having a plurality of arms that can approach and perform operation at regions, organs, tissues and objects having varying sizes while still avoiding larger cuts and ensuring lesser trauma to patient, less post-operative pain and faster recovery of the patient.

Another object of the present disclosure is to provide a robotic surgical instrument system having a plurality of arms that facilitates single port surgery and therefore ensures various benefits of single port surgery that includes better cosmetic results for patients, less blood loss and easy tissue retrieval.

Yet another object of the present disclosure is to provide a robotic surgical instrument system having a plurality of arms that maintains benefits of single port surgery while reducing a surgeon's fatigue that is prevalent in the case of manual single port surgeries.

Still a further object of the present disclosure is to provide arms for a robotic surgical instrument system that achieve movement along at least seven axes of movement, thereby enabling a surgeon to perform complex procedures even while operating through a miniaturized single port.

Another object of the present disclosure is to provide an arm for a robotic surgical instrument system that can be used together along with another arm to define a dual arm configuration of a robotic surgical instrument system and enabling the arms to approach and access a region, an organ, an object or a tissue inside an operation site from opposite sides, thereby enabling the arms to simultaneously approach both sides of a large as well as a small organs, objects and tissues like tumors and facilitating better control during a surgical procedure.

Another object of the present disclosure is to provide arms for a robotic surgical instrument system that has such configuration that enables the arms to be inserted straight inside an operation site though a miniature aperture configured on a single access port mounted on an incision on a patient's body at the operation site and to be moved inside the operation site to ensure access to organs in the operation site.

Yet another object of the present disclosure is to provide arms for a robotic surgical instrument system that operates in pair and that can be configured inside an operation site to ensure approach to organs from opposite sides, thereby enabling the articulating arms to simultaneously approach both sides of a larger organ or objects like tumors.

Still a further object of the present disclosure is to provide arms for a robotic surgical instrument system that can be displaced remotely, thereby enabling the arms to be inserted straight into an abdominal cavity, thereafter be moved inside the abdominal cavity to ensure convenient approach by achieving movement along more axes and access to the organs by enhancing reach of the arms.

Another object of the present disclosure is to provide a robotic surgical instrument system having a plurality of arms, wherein the robotic arms thereof can be conveniently and remotely controlled using joysticks and as such ensures ergonomic posture for the surgeon, thereby reduces surgeon's fatigue and chances of error due to fatigue.

Another object of the present disclosure is to provide arms for a robotic surgical instrument system, wherein elements configuring the arms are connected to each other by pitch joints, thereby facilitating triangulation of forces, enhancing leverage and providing actuating forces at the distal end of the arms to enhance maneuverability of the arms without requiring a lot of movements outside a patient's body above an access port mounted on an incision formed on the patient's body, wherein such movements outside the patient's body can be uncomfortable and unsafe for the surgeon and the people working in close proximity of patient and without need for repeatedly starting and stopping the operation.

Still another object of the present disclosure is to provide a robotic surgical instrument system having a plurality of arms that reduces exposure of internal organs to possible external contaminants thereby reducing risks of acquiring infections.

Furthermore, object of the present disclosure is to provide a robotic surgical instrument system having a plurality of arms that provide approach similar to that of multiport surgery such that the organs could be approached just like multiport surgery but through single port, thereby reducing the learning curve of the surgeons.

Yet another object of the present disclosure is to provide arms for a robotic surgical instrument system that does not exert any lateral forces on an access port mounted on an incision formed on the patient's body and the patient's abdomen wall.

Another object of the present disclosure is to provide arms for a robotic surgical instrument system that has such configuration that the roll, pitch and finger movement are provided at distal end thereof, thereby enabling control of the articulating arm from proximal end.

Yet another object of the present disclosure is to provide arms for a robotic surgical instrument system that are independent of each other, thereby facilitating convenient and quicker tool change or functionality (grasper etc.) change as only one arm is required to be retracted as opposed to retracting the entire bundle from the patients body in case of conventional robotic surgical instrument system.

Another object of the present disclosure is to provide a method for performing single port robotic surgery that utilizes arms that are capable of linearly entering into an operation site and can be moved inside the operation site for achieving movement along more axes and enabling the arms to perform the surgical procedure.

### SUMMARY

An articulating arm for a robotic surgical instrument system for performing single port procedures is disclosed in accordance with an embodiment of the present disclosure. The articulating arm includes at least a pair of arm elements, an elongate support element, a pair of end effectors and a wrist joint. The pair of arm elements, particularly a first arm element and a second arm element of the pair of arm elements are joined to each other by at least one first pitch joint. The elongate support element is connected at one end thereof to the first arm element by at least one second pitch joint and other end of the elongate support element is connected to a support structure and is functionally coupled to a control of the robotic surgical instrument system. The pair of end effectors are connected to each other by means of a hinge joint, wherein the hinge joint facilitates swiveling of the end effectors relative to each other to configure scissoring action of the end effectors. The wrist joint connects the pair of end effectors to the distal end of the second arm element, wherein the wrist joint facilitates yaw movement and rolling movement of the end effectors. The elongate support element, the arm elements, the pitch joints and the end effectors are aligned linearly in one operative configuration for insertion via an aperture of a port into an operation site and once inserted are articulated about the pitch joints, and moved with the help of the other joints to perform a single port procedure with at least seven axes of movement.

Typically, each pitch joint includes a pair of pitch base members and an axis member, wherein the pair of pitch base members extend from the first arm element and are spaced from each other and the axes member is pivotably supported between the pitch base members and supports thereon the second arm element.

Generally, the axis member is a yaw axis member.

In a first operative configuration of the articulating arm, the arm elements, the pitch joints and the end effectors are linearly aligned and move along a linear axis to define movement of the articulating arm about a first linear axis of movement. In a second operative configuration of the articulating arm, the first arm element moves with respect to the elongate support element to define movement of the articulating arm about a second axis to define a first pitch motion. In a third operative configuration of the articulating arm, the second arm element and the first arm element move with respect to each other to define movement of the articulating arm about a third axis to define a second pitch motion. In a fourth operative configuration of the articulating arm, the end effectors fitted to the distal end of the second arm element via the wrist joint rolls either in linear and non-linear configuration of the articulating arm to define movement of the articulating arm about a fourth axis to define wrist roll motion. In a fifth operative configuration of the articulating arm, the end effectors are fitted to the distal end of the second arm element via the wrist joint and is able to yaw about the wrist joint to define movement of the articulating arm about a fifth axis to define a wrist yaw motion. In a sixth operative configuration of the articulating arm, the elongate support element of the articulating arm swivels sideways for defining movement of the articulating arm about a sixth axis of movement. In a seventh operative configuration of the articulating arm, the support element of the articulating arm swivels by +/−10 degrees with respect to a port entry point to create volumetric work envelop and define the movement of the articulating arm about a seventh axis. In an eighth operative configuration of the articulating arm, the end effectors are swiveled independently with respect to each other about the hinge joint to provide either of gripping and cutting force to the end effectors.

A robotic surgical instrument system for performing surgical procedures is disclosed in accordance with an embodiment of the present disclosure. The robotic surgical instrument system includes a pair of articulating arms as claimed in claim 1, a control and a resilient access port. The control independently controls the movements of each of the articulating arms, the resilient access port has at least three apertures and is mounted on an incision on a patient's body at the operation site, wherein a pair of apertures of the at least three apertures receives the pair of articulating arms for performing a single port procedure on a site corresponding to the incision.

The robotic surgical instrument system further includes at least one input device, a visioning system and at least one output device. The least one input device co-operates with the control to remotely manipulate the articulating arms by controlling each of the movements of the arm elements of the articulating arms and yawing, rolling and swiveling movements of the end effectors of the articulating arms. The visioning system has a camera element that is introduced through an aperture of the port for visioning said operation site. The at least one output device displays images captured by the visioning system.

Typically, the at least one input device is selected from a group consisting of a joystick, a touch-screen and a foot control pedal.

A method for performing a robotic surgery is disclosed in accordance with an embodiment of the present disclosure. The method includes the steps of linearly introducing, via apertures of a resilient access port, into an operation site where a surgery is required to be performed, a pair of articulating arms, wherein each articulating arm includes a first arm element and a second arm element of at least a pair of arm elements joined to each other by at least a first pitch joint, an elongate support element joined to the first arm element by at least a second pitch joint and a pair of end effectors connected to each other by a hinge joint and to the distal end of the second arm element via a wrist joint, thereafter, articulating the arm elements in a controlled manner, with respect to each other to approach and access regions, organs, tissues and objects inside the operation site and achieve triangulation of forces to perform operation at regions, the organs, tissues and objects while still requiring proportionately less actuation forces to generate a predetermined horizontal pull force and rolling, yawing and swiveling of the end effectors to facilitate performing of a single port procedure.

Generally, the method for performing a robotic surgery includes the step of controllably skewing the elongate support elements corresponding to each of the arms to relatively displace the arm elements away from each other.

Typically, the method for performing a robotic surgery includes the step of controlling the movements of the arms to achieve triangulation of forces at the end effectors to perform operation at regions, organs, tissues and objects, and generate a horizontal pull force.

Preferably, the method for performing a robotic surgery includes the step of displacing the support elements in an operative configuration between a first arrangement wherein the support elements are parallel to each other, and a second arrangement wherein the support elements are skewed with respect to each other, wherein in the second arrangement the support elements can be configured to be inclined away from each other or inclined crossing to each other with pivot at the access port.

In accordance with an embodiment, the elongate support element can be angularly displaced with respect a support structure by an angle between −30 degrees and +30 degrees.

In accordance with another embodiment, the first arm element can be angularly displaced with respect to the elongate support element by an angle between −60 degrees and +60 degrees and the second arm element can be angularly displaced with respect to the first arm element by an angle between −60 degrees and +60 degrees.

### BRIEF DESCRIPTION OF THE ACCOMPANYING DRAWINGS

The foregoing features of the present invention will become more apparent from the following description and appended claims, taken in conjunction with the accompanying drawings. Understanding that these drawings depict only typical embodiments of the invention and are, therefore, not to be considered limiting its scope, the invention will be described with additional specificity and detail through use of the accompanying drawings in which:

FIG. 1 illustrates an isometric view of a robotic surgical instrument system in accordance with the present invention;

FIG. 2 illustrates the insertion of surgical arms of the system of FIG. 1 through an access port;

FIG. 3 illustrates an isometric view of the movement of tools at the end of the surgical arms of the system of FIG. 1, wherein the tools approach is similar to approach in case of multi-port surgery;

FIG. 4 illustrates an end view of the movement of tools in an operative space via an access port;

FIGS. 5 to 10 illustrate the system in accordance with the present invention under various operative configurations, particularly, FIG. 9 and FIG. 10 illustrates a camera, along with an umbilical cord and a magnet used for securely holding the camera;

FIG. 11 is a cross sectional view of a pair of surgical arm mounting robots and associated surgical arms of the system of FIG. 1;

FIG. 12 is an isometric view illustrating details of one surgical arm of the system of FIG. 1,

FIG. 13 is an isometric view of motor mounting, pitch-1 base and pitch-1 axis that form part of a surgical arm of the system of FIG. 1;

FIG. 14 is an exploded view of FIG. 13;

FIG. 15 is an isometric view of the wrist joint depicting the point of connection between the wrist joint and the pitch joint that form part of a surgical arm of the system of FIG. 1;

FIG. 16 is an exploded view of pitch joint of FIG. 15;

FIG. 17 is an isometric view of an arm wrist and yaw assembly that form part of a surgical arm of the system of FIG. 1; and

FIG. 18 is an exploded view of FIG. 17;

FIG. 19 illustrates a schematic representation of a pair of articulating arms in accordance with another embodiment of the present disclosure entering an abdominal cavity along with a vision system via a single port mounted on an incision formed on a patient's body, wherein the articulating arms are inserted straight into the abdominal cavity, thereafter are moved inside the abdominal cavity;

FIG. 20a-FIG. 20c illustrates different views of an articulating arm of FIG. 19, wherein arm elements configuring the articulating arm are moved and a finger formation disposed at the distal end of the articulating arm is moved for enabling the articulating arm to achieve movement along at least seven axes of movement;

FIG. 21a and FIG. 21b illustrates a schematic representation of a conventional surgical arm and an articulation arm in accordance with an embodiment of the present disclosure, wherein the conventional surgical arm has an angle of approach is in the range of 5-10° whereas in case of the articulation arm, the arm elements move with respect to each other and the articulating arm has an angle of approach is in the range of 30-60°;

FIG. 21c illustrates a force triangulation diagram depicting relation between horizontal pull force and pull component along the arm as a function of angle of approach;

FIG. 22a illustrates a schematic representation depicting a front view of a pair of articulating arms entering an abdominal cavity via a single port having two apertures and mounted on an incision formed on a patient's body in accordance with an embodiment, wherein the articulating arms are inserted straight into the abdominal cavity, thereafter are moved inside the abdominal cavity to simultaneously approach both sides of a comparatively smaller organ “Os”, tissues and objects;

FIG. 22b illustrates a schematic representation depicting a front view of a pair of articulating arms entering an abdominal cavity via a single port having two apertures and mounted on an incision formed on a patient's body in accordance with another embodiment, wherein the articulating arms are inserted straight into the abdominal cavity, thereafter are moved inside the abdominal cavity to simultaneously approach both sides of a comparatively larger organ “O_{L}”, tissues and objects; and

FIG. 22c illustrates a schematic representation depicting a side view of the pair of articulating arms that are moved inside the abdominal cavity in a skewed configuration with pivot point at the access port to simultaneously approach both sides of a comparatively larger organ “O_{L}”, tissues and objects in a different plane.

### DETAILED DESCRIPTION

The present disclosure will now be described with reference to the accompanying drawings which do not limit the scope and ambit of the disclosure. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The systems known in the art are plagued by drawbacks including a need to provide multiple and larger incisions in the patient's body, risks of infection and lesions and a longer time for healing the multiple and larger incisions that require more time to heal. In accordance with the present disclosure, there is provided an ergonomically designed robotic surgical instrument system suitable for use during laparoscopic surgery to facilitate access to an insufflated abdominal cavity while maintaining pneumoperitoneum. The system comprises at least two external surgical arm mounting robots co-operating with an associated surgical arm that holds tools for performing a surgical procedure. Each surgical arm is provided with at least two articulation joints. The surgical arms are inserted into the operative space in a substantially straight configuration and manipulated by a surgical console to achieve the triangulation of forces in the operative space. The need for a single incision for a single access port and the method of achieving triangulation of forces within the operative space are the main advantages of the present invention that lead to minimum movement of the system outside the surface of the patient's body and minimum invasion, thus overcoming the drawbacks of the prior art.

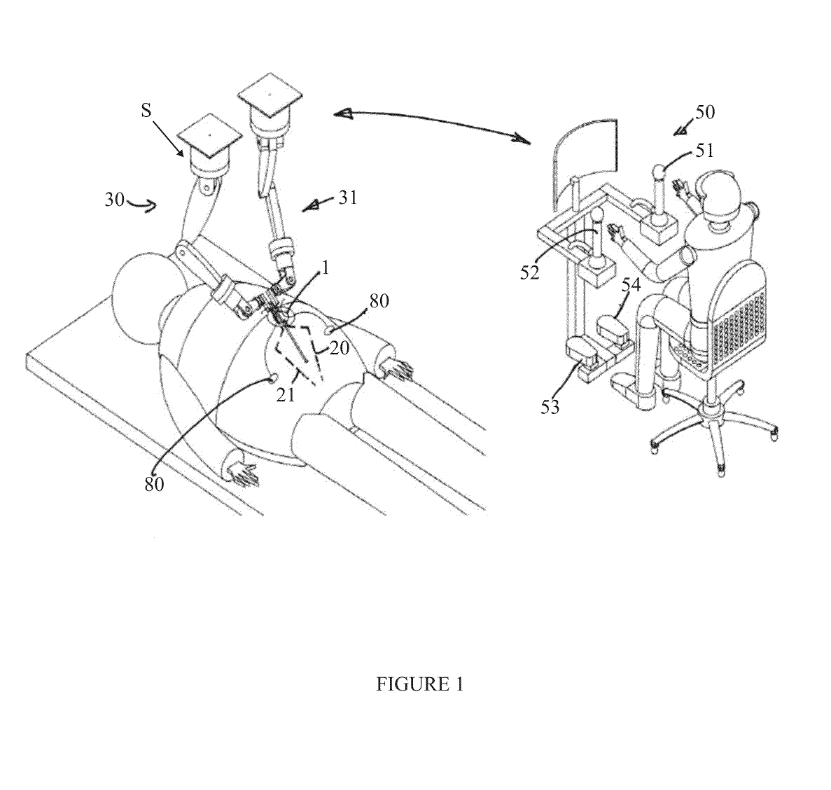

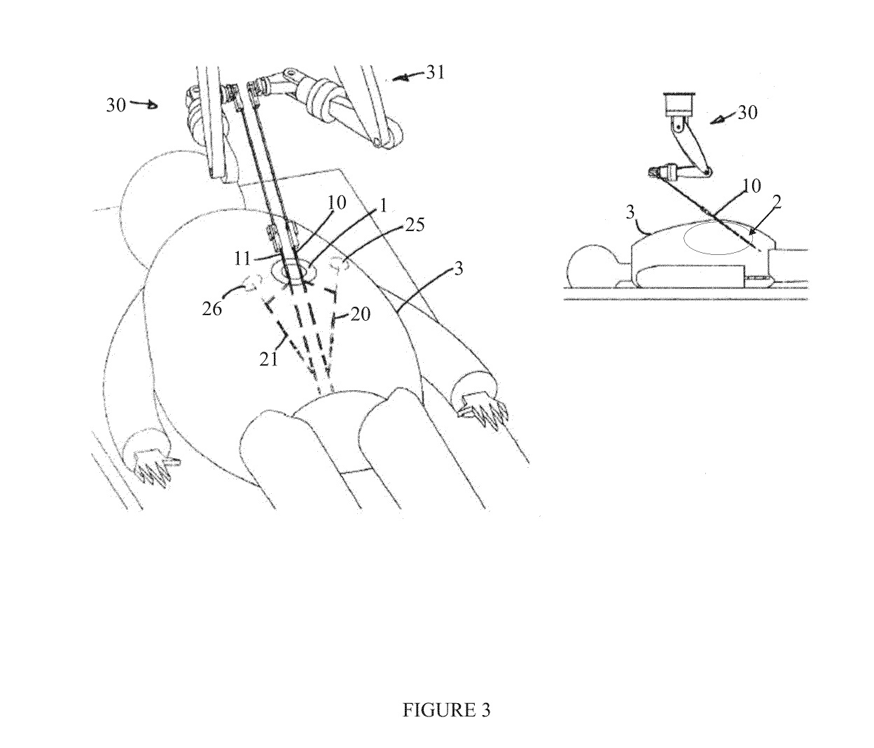

Referring to FIGS. 1 to 3, a robotic surgical instrument system in accordance with the present invention mainly includes two external surgical arm mounting robots 30, 31 and two surgical arms 10, 11 controlled by an external surgical console or control 50 which typically comprises two hand joysticks 51, 52 and foot controls 53, 54 for manipulation of the surgical arms 10, 11, tools 20, 21, position of the mounting robots 30, 31 and a vision system 80. The control independently controls maneuvering of the articulation arms, the control includes at least one input device and at least one output device, wherein the at least one input device remotely manipulates the articulating arms and a pair of end effectors and at least one output device display images captured by a vision system for viewing area of operation of distal ends of the articulating arms for facilitating maneuvering of the articulating arms. The at least one input device can be a hand joystick or a foot control pedal. The output device can be Thin Film Transistor (TFT) display screen or Liquid Crystal Display (LCD) screen.

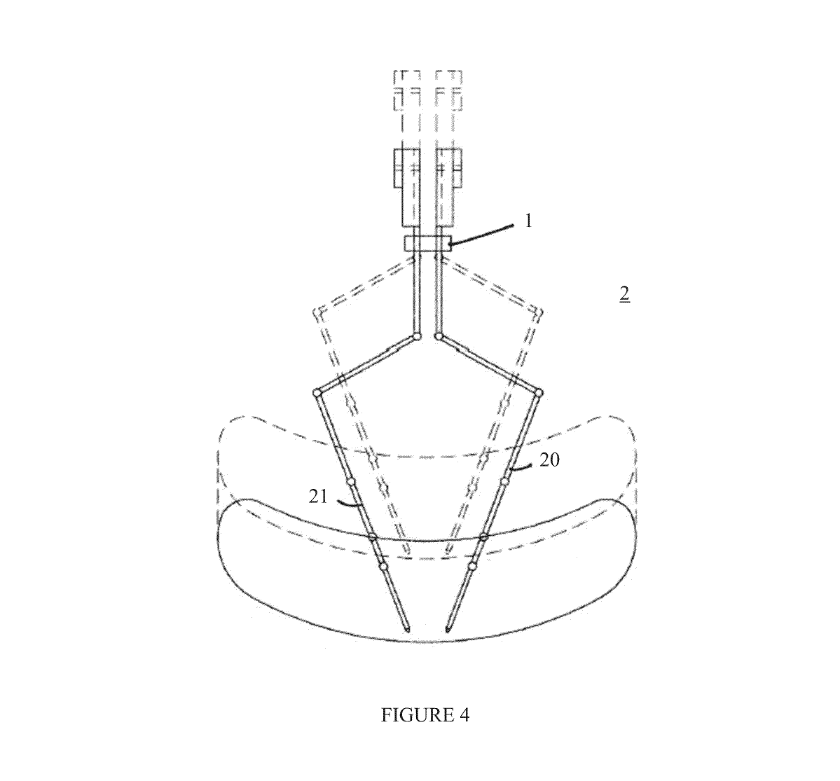

The system in accordance with the present invention is a dual articulation arm configuration robot that enables entry into an operative space 2 in the abdominal cavity via an access port 1 for performing a surgical procedure. The access port 1 is adapted to facilitate unhindered access to the operative space 2. The access port 1 is typically a gel port, a puncturable sealed port or a port with pre-punctured openings. Typically, the access port 1 receives at least two surgical arms 10, 11 and a vision system 80 to be inserted into the operative space 2 via the access port 1. The surgical arms 10, 11 enter the operative space 2 in a substantially straight line, and are then moved inside the operating space 2 within the patient body, “by triangulation” achieved by the surgical console 50. The process of triangulation typically involves determining a precise operative site by measuring angles to it from known points at either end of a fixed baseline, rather than measuring distances to the site directly. The system in accordance with the present invention enables the advantages of “triangulation” as if operating in a biport configuration. The arms operate as if the tools 20, 21 were inserted in biport configuration through “virtual” ports 25, 26 as per established biport procedures. FIG. 4 illustrates an end view of the movement of the tools 20, 21 in the operative space 2 via the access port 1. The preferred embodiment of the present invention requires a single access port 1 for insertion of the surgical arms 10, 11. However, in accordance with an alternative embodiment, the surgical arms are inserted through two discrete access ports.

The two external surgical arm mounting robots 30, 31 are each provided with at least seven degrees of freedom for facilitating positioning of the articulating surgical arms 10, 11 with respect to the patient and the bed setup for the surgical procedure.

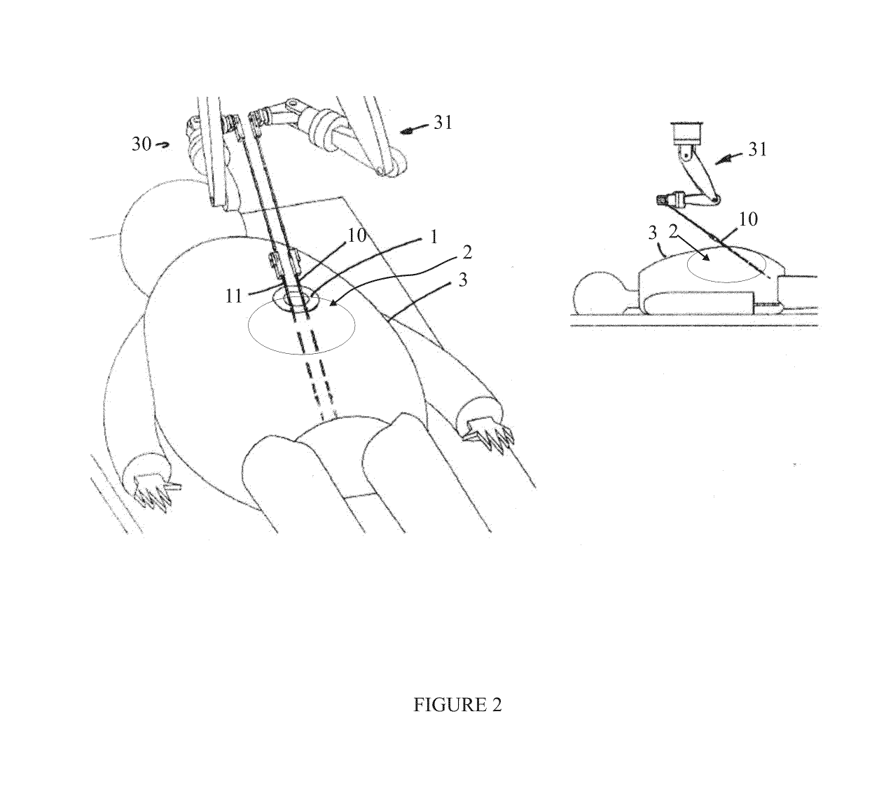

The articulating surgical arm mounting robots 30, 31 enable the X, Y, Z positions and angle of approach to the desired operating site to be achieved in a straight configuration, when surgical arms are inserted as illustrated in FIG. 2. These robots can be floor mounted or ceiling mounted—freeing up the space around the patient for surgeons and assistants.

The system in accordance with the present invention provides a sufficiently large work envelope that enables precision manipulation required for surgical procedures inside the patient's body without significant motion outside the patient's body. This frees up external space, and allows safe operative space for the surgeons/assistants around the robotic system, without keeping a side of the patient occupied by a large moving floor—mounted structure.

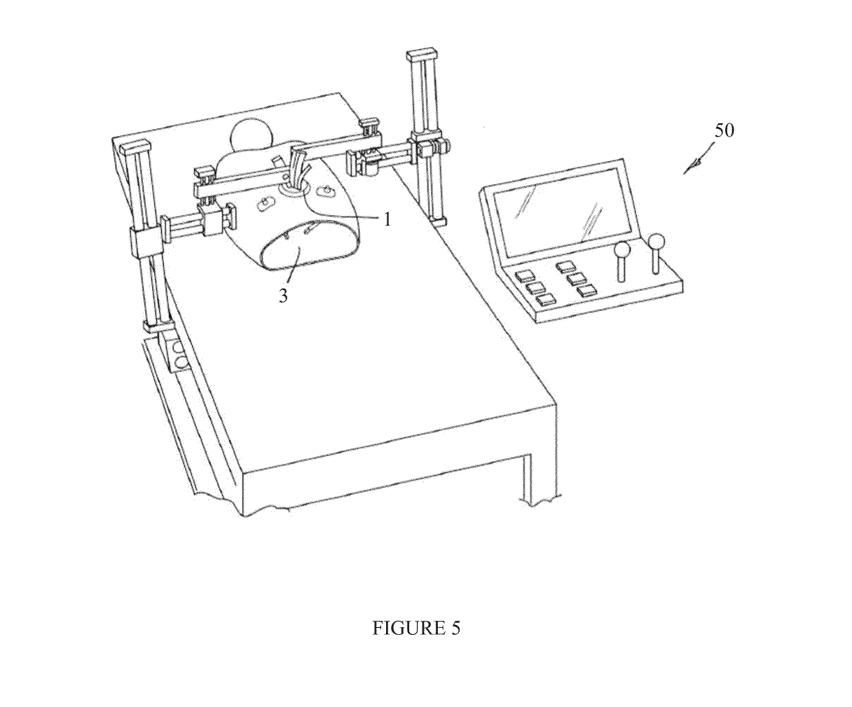

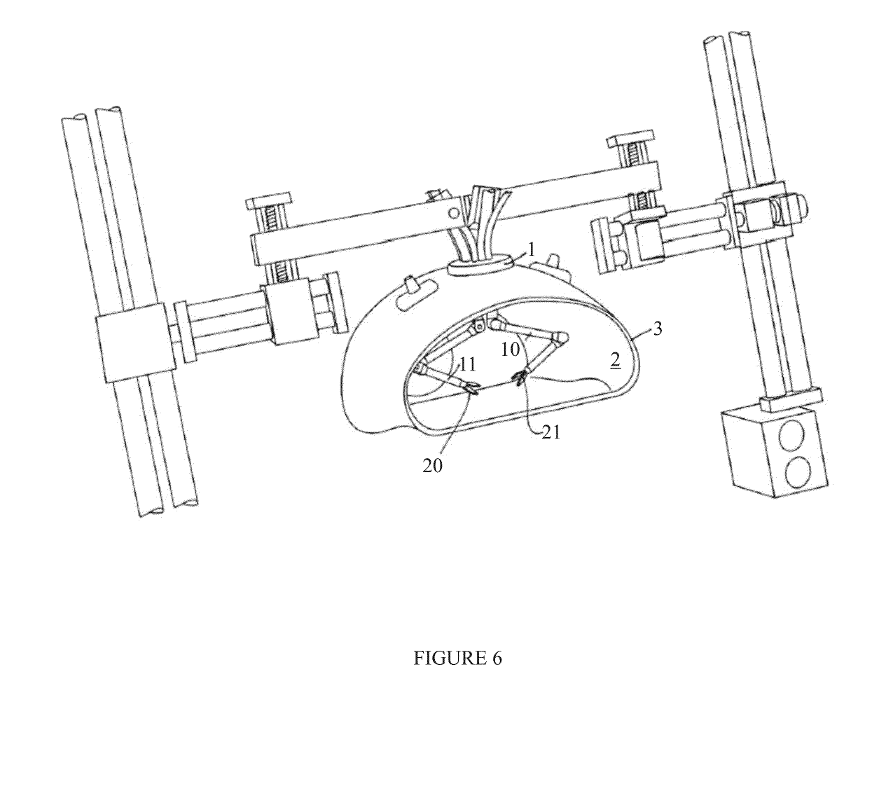

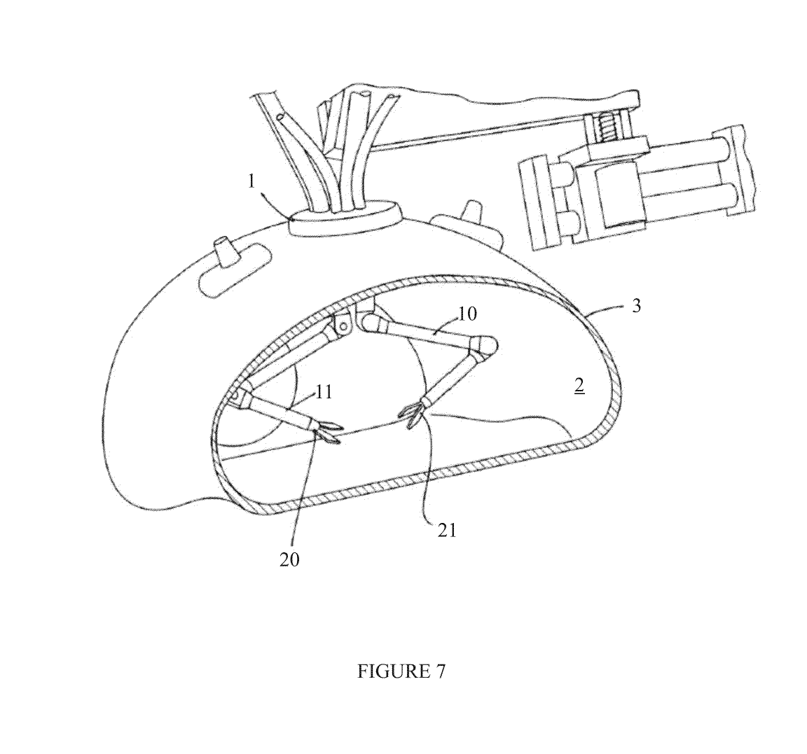

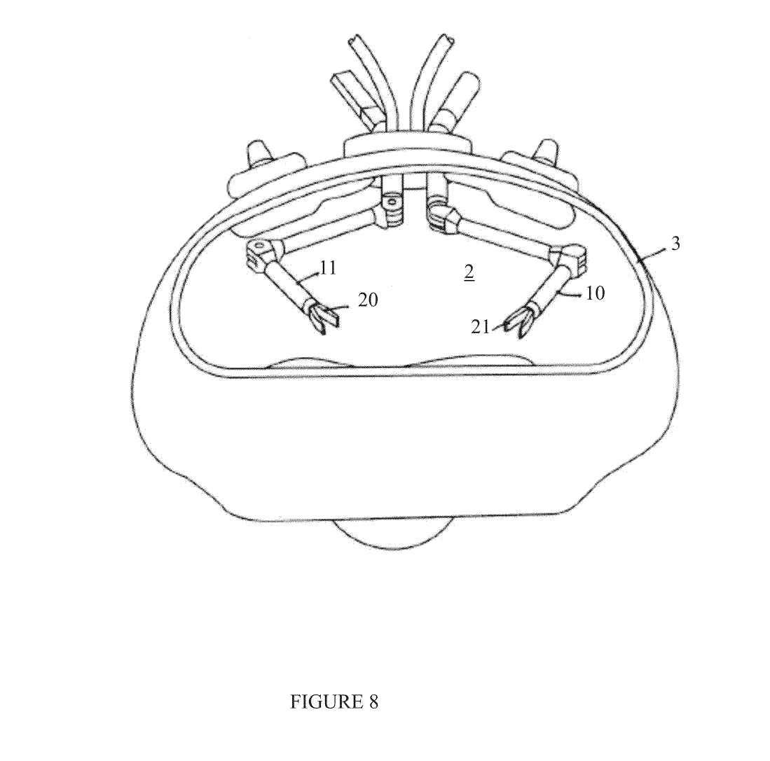

FIGS. 5 to 10 illustrate the system in accordance with the present invention under various operative configurations.

Tools 20, 21 at the end of the surgical arms 10, 11 are attached on or detached from the surgical arms 10, 11 either inside or outside the operative space 2. In one embodiment of the present invention, tools are attached to the surgical arm before insertion of the surgical arm through the access port 1. Alternatively, in accordance with another embodiment, tools are attached to the surgical arm after insertion of the surgical arm through the access port 1. The tool change is performed within the operative space 2 without a requirement to extract the surgical arm fully out, through a separate assistant port (not shown).

The movement of the surgical arms 10, 11 is controlled using a mechanism of cables, pulleys and linkages, configured such that actuation is always achieved by the cables in tension, resulting in precision motion.

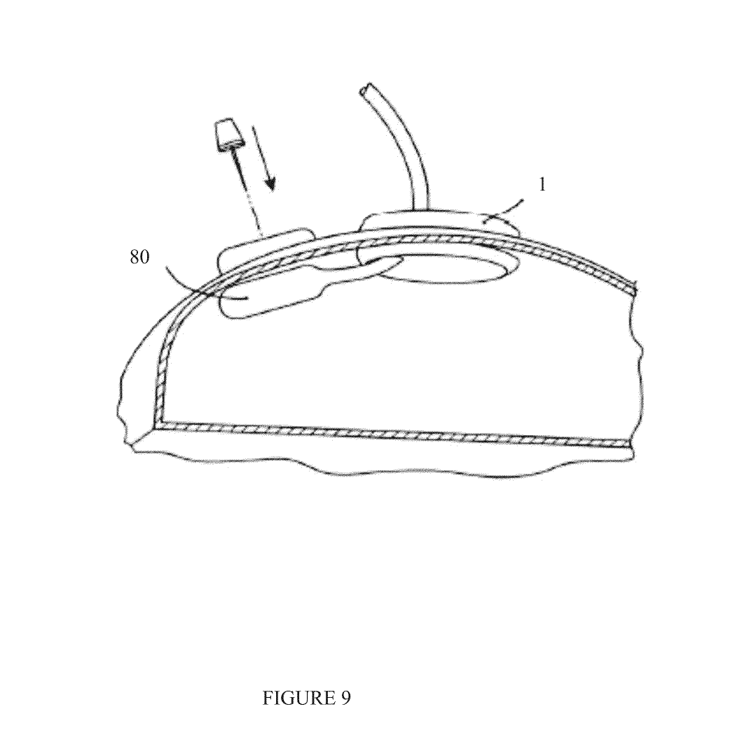

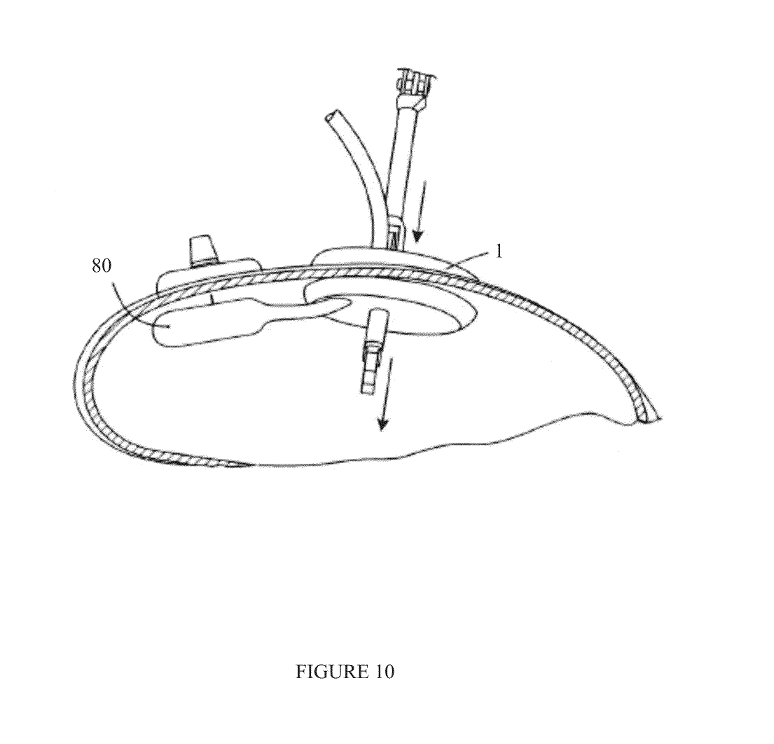

The system in accordance with the present invention further comprises at least one visioning system, also referred to as a vision system. The vision system is typically a fiber optic scope, an insertable camera system, or a separate insertable camera 80 through an “umbilical chord” cable inserted through the same access port 1 or optionally, another access port (not shown). The camera is anchored to the abdominal wall as illustrated in FIGS. 9 and 10. Preferably, a magnet is used to hold the camera to the abdominal wall. Alternatively, to provide enhanced visibility within the operative space 2, two such cameras 80 or vision systems are provided.

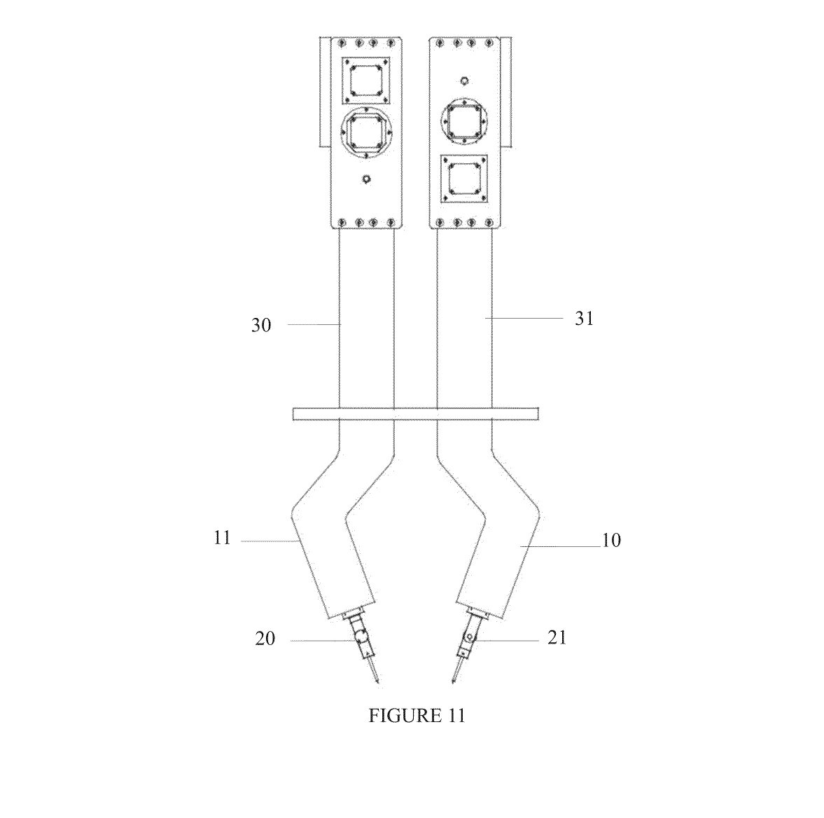

Mechanical details of the construction of the robotic system in accordance with the present invention are illustrated in FIGS. 11 to 18.

Referring to FIG. 11, each of the surgical arm mounting robots 30 and 31 are provided with a motor (not specifically referenced) at each of the articulation joints thereof, wherein each motor facilitates rotation of a pulley which in turn results in tension in the associated cables; the tension in the cables facilitates the movement of the surgical arms 10, 11.

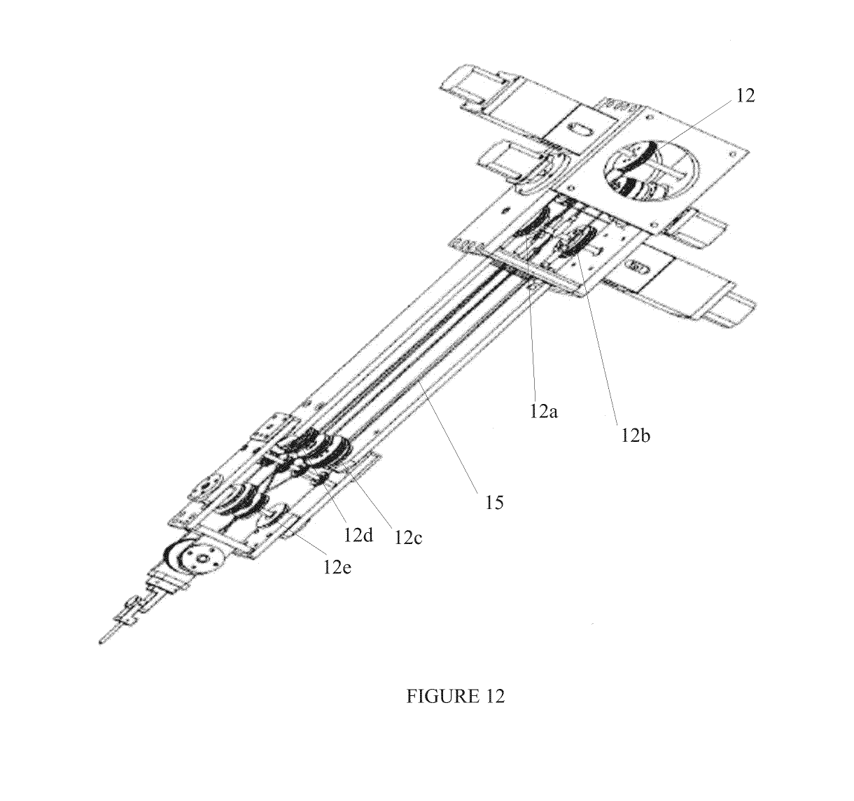

Referring to FIG. 12 of the accompanying drawings, a motor (not specifically referenced) is provided for driving a pulley 12. A cable 15 passes over the pulley 12 and imparts required motion to the surgical arms 10, 11. Further, there are plurality of idler pulleys 12a-12e provided for tensioning cables represented by cable 15 and resulting in precision motion of the surgical arms 10, 11.

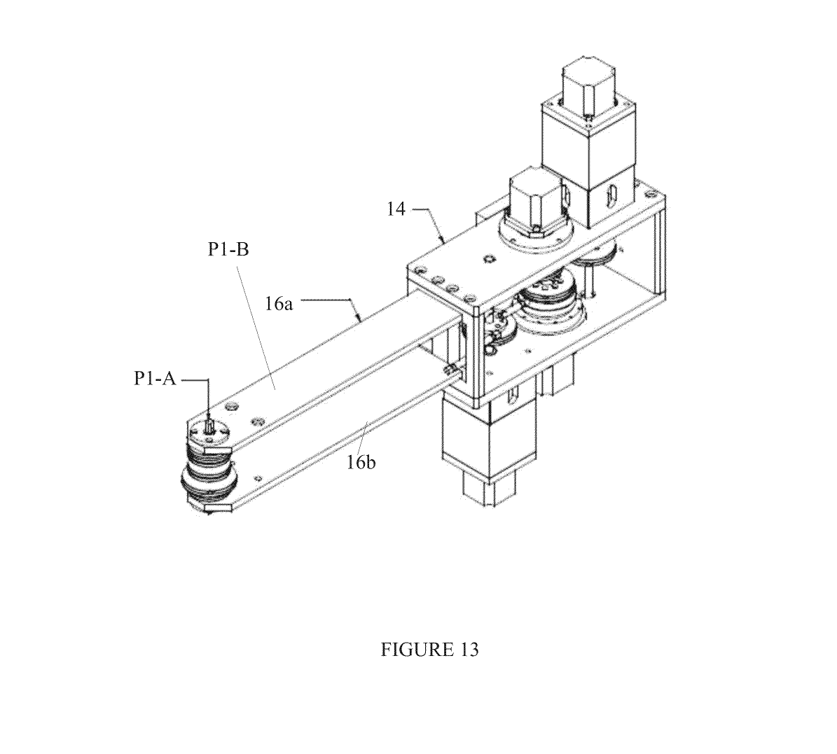

Referring to FIG. 13 of the accompanying drawings, the motor (not specifically referenced) as well as the pulley 12 (shown in FIG. 12) driven by the motor are both housed inside a motor mounting 14. A pitch-1 base P1-B in the form of spaced apart plates 16a and 16b extends outwardly from the motor mounting 14. A pitch-1 axis P1-A is located at the distal end of the pitch-1 base P1-B. More specifically, the arm elements of the articulating arm are joined by pitch joints, wherein each pitch joint includes a pair of pitch base members P1-B (illustrated in FIG. 13) and an axis element P1-A (illustrated in FIG. 13), wherein the pair of pitch base members P1-B extends from a first element 14 configuring the articulating arm and are spaced from each other and the axis element P1-A is pivotably supported between the pitch base members P1-B and supports thereon a second arm element (not illustrated in FIG. 13) disposed adjacent to the first element 14 and configuring the articulating arm. In accordance with an embodiment the axis element is a yaw axis element.

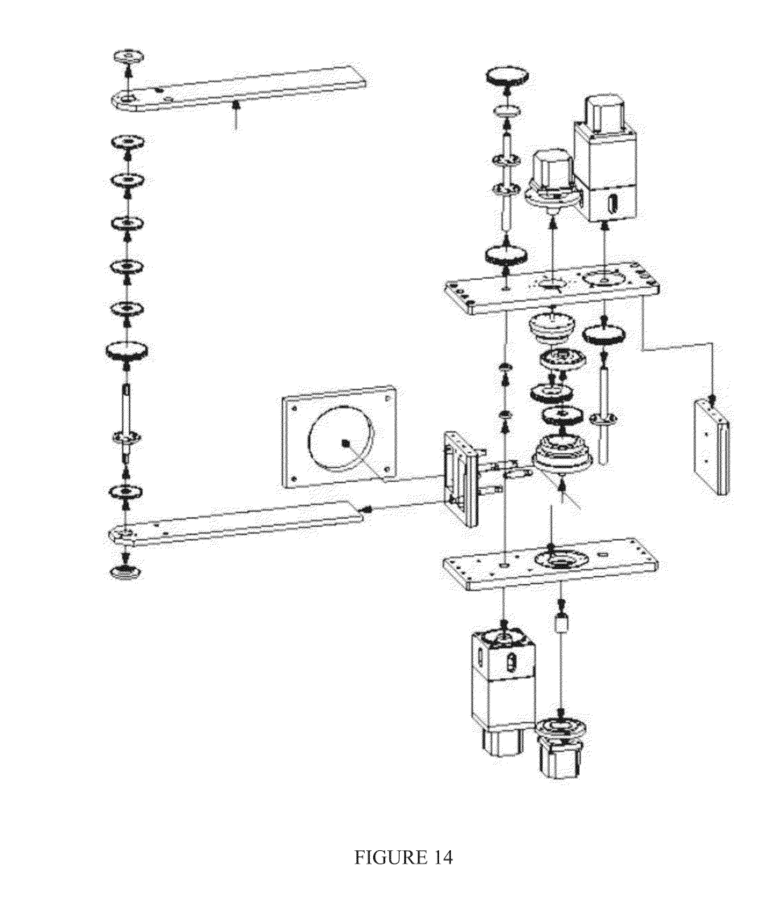

Referring to FIG. 14, the motor mounting 14 comprises a plurality of plates assembled together by a plurality of fastening elements for securely holding the motor and the pulleys therein.

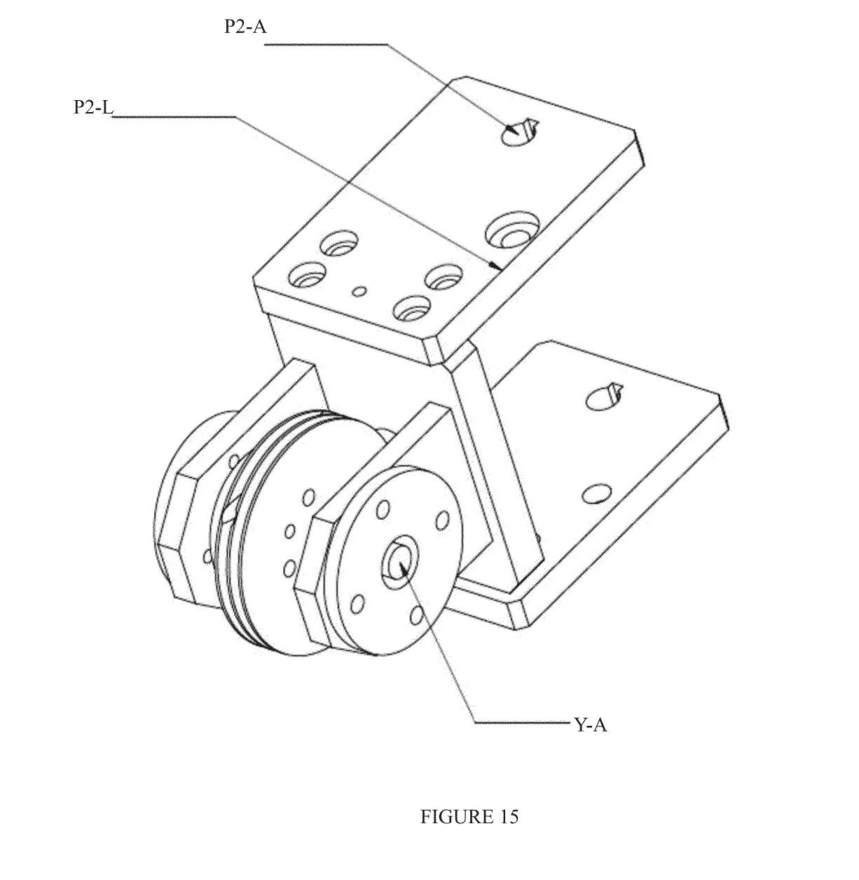

FIG. 15 is an isometric view of a pitch link P2-L, pitch axis P2-A and yaw axis Y-A that form part of a surgical arm of the system of FIG. 1.

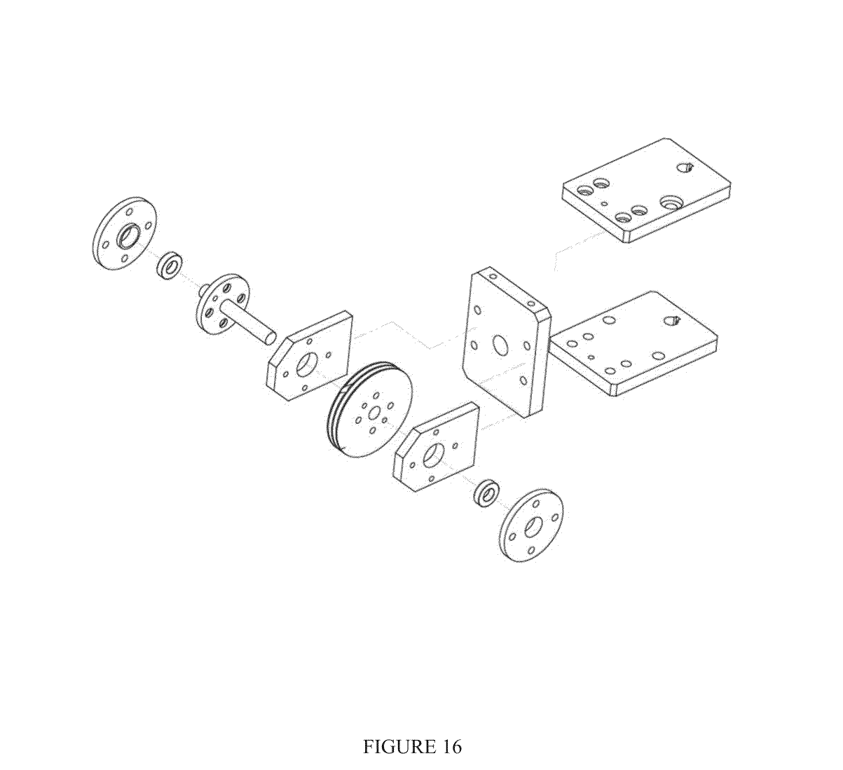

FIG. 16 is an exploded view of the pitch joint of FIG. 15.

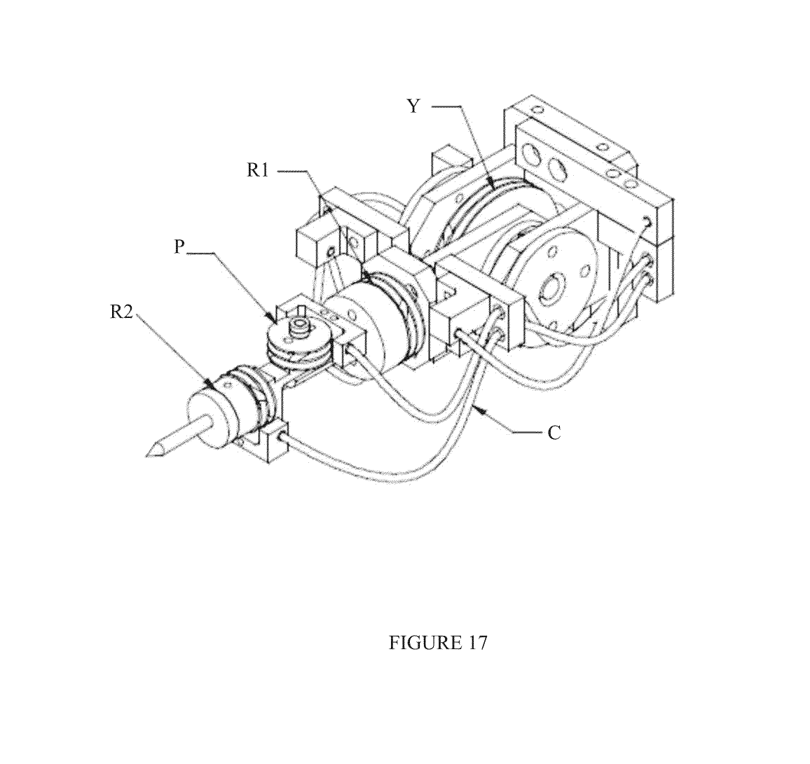

FIG. 17 is an isometric view of an arm wrist and yaw assembly that forms part of a surgical arm of the system of FIG. 1. a first roll, a second roll, pitch, yaw and the co-axial driving cables being referenced generally by the alphanumeric characters namely R1, R2, P, Y, and C respectively.

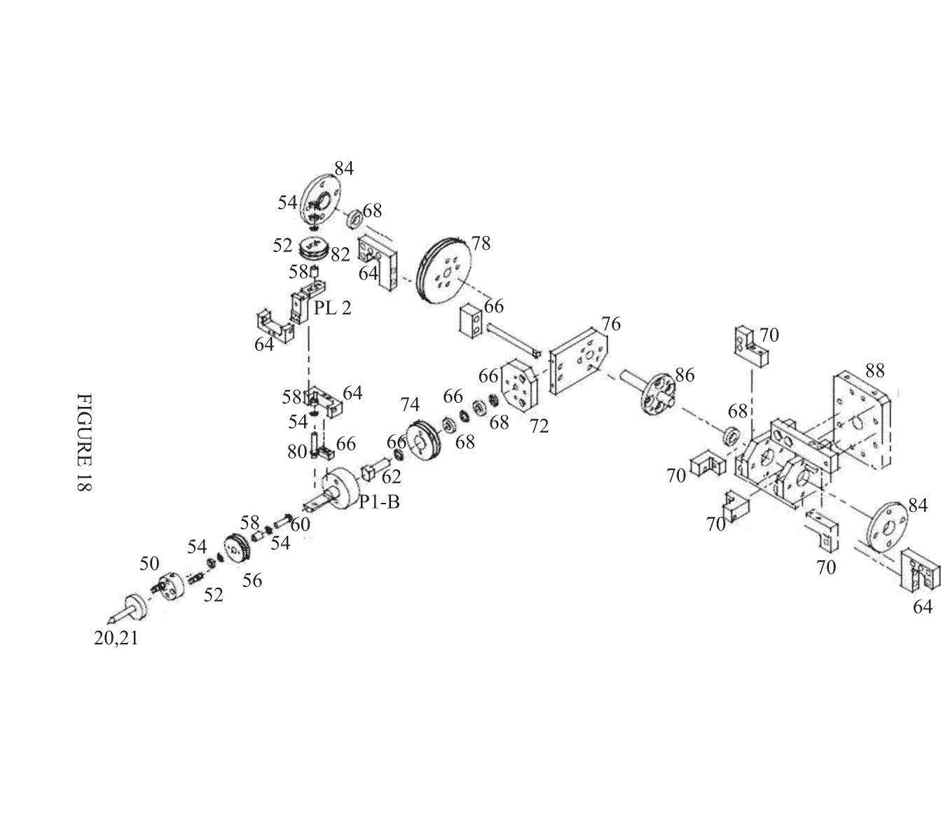

FIG. 18 is an exploded view of FIG. 17 and the key components are referenced generally as follows:
tool 20, 21;
tool holder 50;
nut 52;
teflon washer 54;
roll 2 pulley 56;
bush 58;
roll 2 shaft 60;
roll 1 shaft 62;
co-axial driving cable mount 64;
pitch base members P1-B;
spacer 66;
bearing 68;
co-axial driving cable bracket 70;
roll base 72;
roll 1 pulley 74;
yaw link 76;
yaw pulley 78;
pitch shaft 80;
pitch pulley 82;
bearing cap 84;
yaw shaft 86;
back plate 88; and
pitch link P2-L.

The articulating arm for the robotic surgical instrument system includes at least a pair of arm elements and a pair of end effectors. The arm elements are joined to each other by pitch joints. More specifically, a first arm element of the pair of arm elements is connected to an elongate support element, also referred to as a support element via at least one second pitch joint, the first arm element and a second arm element of the pair of arm elements are joined to each other by at least one first pitch joint. In accordance with one embodiment, the arm elements are rigid. The end effectors are connected to the distal end of the second arm element via a wrist joint, wherein the wrist joint facilitates yaw movement and rolling movement of the end effectors. The end effectors are connected to each other via a hinge joint, wherein the hinge joint facilitates swiveling of the end effectors relative to each other to configure scissoring action of the end effectors. With such configuration the end-effector can be used for gripping a surgical tool such as a surgical knife or for operating another surgical tool such as a surgical scissor that require scissoring action. In accordance with another embodiment, any other surgical tool can be removably connected to the distal end of the second arm element depending upon requirements and application. The arm elements, the pitch joints and the end effectors are arranged linearly in one operative configuration and are linearly introduced into an operation site through one of the plurality apertures configured on an access port mounted on an incision on a patient's body at the operation site such that at least one of the pitch joints is inside the patient's body, the arm elements move with respect to each other about the pitch joints to configure a second operative configuration thereof. The end effector fitted at the distal end of the arm via the wrist joint yaws, rolls and swivels about the wrist joint to facilitate performing of a surgical procedure. The proximal end of the articulating arm is configured to be connected to a control which enables articulation of the arm elements, and yawing, rolling and swiveling movements of the end effectors to achieve movement along at least seven axes of movement of the arm. The articulating arms are inserted into the operative space via a single access port in a substantially straight configuration and are manipulated by a surgical console, particularly the control using triangulation in the operative space. The articulation arms that require a single incision and a single access port, the articulating arms that achieves movement along at least seven axes of movement for enabling a surgeon to perform complex procedures while operating through a miniaturized single port and still maintaining proper posture and the method of achieving triangulation within the operative space are the main advantages of the articulating arm of the present disclosure that lead to minimum fatigue of the surgeon, thus overcoming the drawbacks of the prior art.

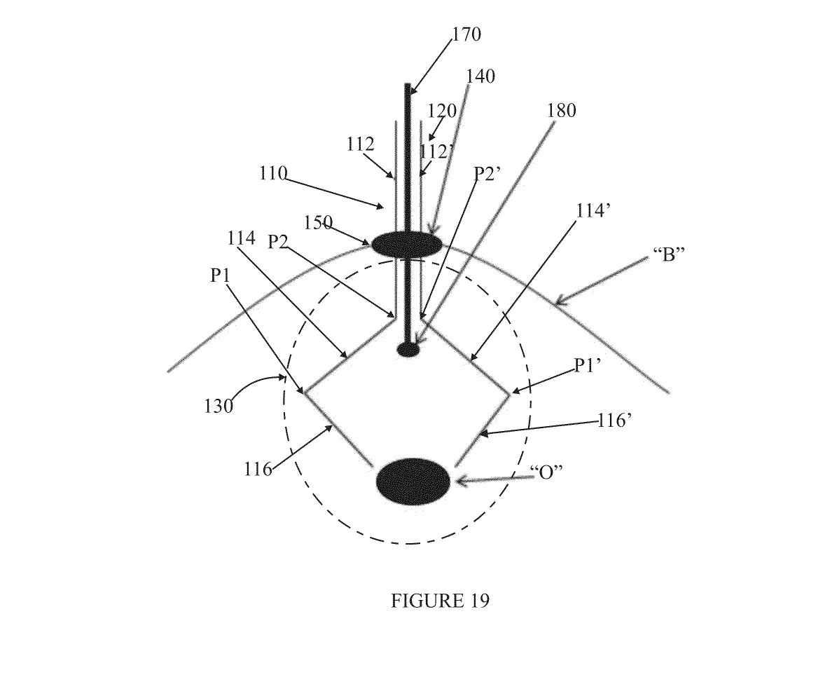

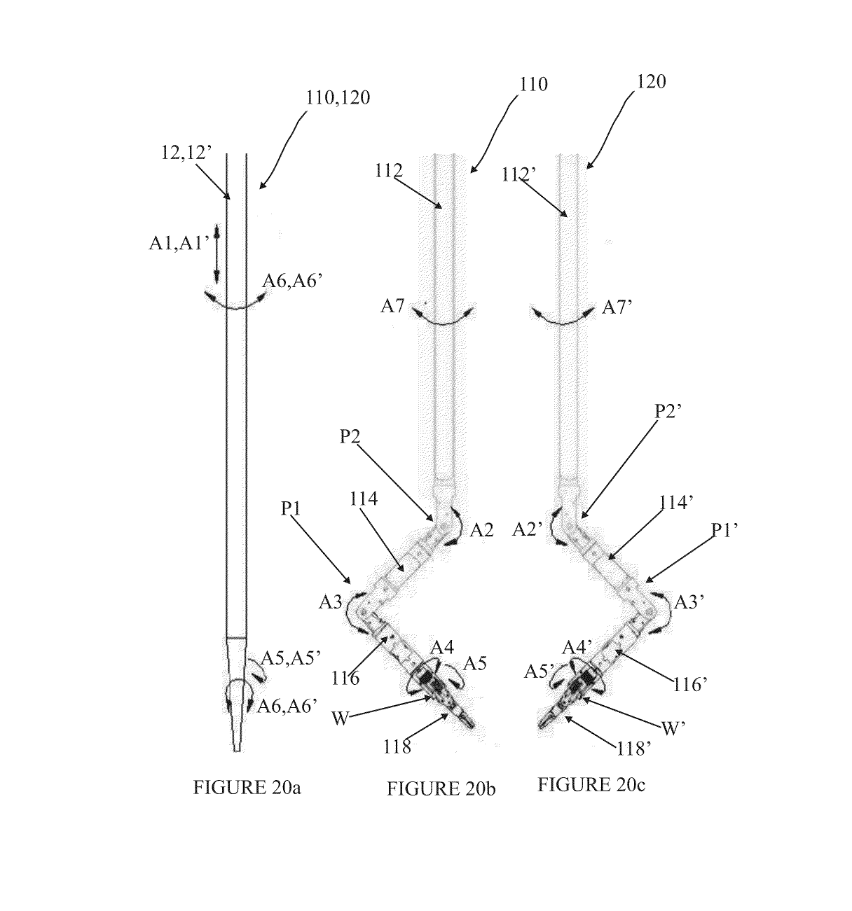

FIG. 19 illustrates a schematic representation of a pair of articulating arms 110, 120 in accordance with another embodiment of the present disclosure entering an operative space, particularly, an abdominal cavity 130 along with a visioning system 170 via apertures configured in an access port 140 mounted on an incision 150 formed on a patient's body “B”, particularly abdomen that is inflated with gas, wherein the articulating arms 110, 120 are inserted straight into the abdominal cavity 130, and are thereafter moved inside the abdominal cavity 130. FIG. 20a-FIG. 20c illustrates different views of an articulating arm 110,120, particularly, FIG. 20a illustrates a side view of either of the articulating arms 110, 120, FIG. 20b illustrates a front view of the articulating arm 110 and FIG. 20c illustrates a front view of the articulating arm 120. Referring to FIG. 19 and FIGS. 20a-20c, the articulating arm 110 of the articulating arms 110, 120 includes at least a pair of arm elements 114 and 116, an elongate support element 112 (also referred to as a support element 112), a pair of end effectors 118 (illustrated in FIG. 20b) and a wrist joint W (illustrated in FIG. 20b). The pair of arm elements 114 and 116, particularly a first arm element 114 and a second arm element 116 of the pair of arm elements 114 and 116 are joined to each other by at least one first pitch joint “P1”. The elongate support element 112 is connected at one end thereof to the first arm element 114 by at least one second pitch joint“P2” and other end of the elongate support element 112 is functionally coupled to a control of the robotic surgical instrument system. More specifically, the elongate support element 112 is connected to a support structure and is angularly displaceable with respect to the support structure to facilitate skewing of the support element about the access port 140. In accordance with an embodiment the elongate support element 112 can be angularly displaced with respect to the support structure by an angle between −30 degrees and +30 degrees. Each pitch joint P1 and P2 includes a pair of pitch base members and an axis member, wherein the pair of pitch base members extend from the first arm element 114 and are spaced from each other and the axes member is pivotably supported between the pitch base members and supports thereon the second arm element 116. Generally, the axis member is a yaw axis member. The pair of end effectors 118 are connected to each other by means of a hinge joint, wherein the hinge joint facilitates swiveling of the end effectors 118 relative to each other to configure scissoring action of the end effectors 118. The wrist joint “W” connects the pair of end effectors 118 to the distal end of the second arm element 116, wherein the wrist joint 116 facilitates yaw movement and rolling movement of the end effectors 118. The elongate support element 112, the arm elements 114 and 116, the pitch joints “P1” and “P2” and the end effectors 118 are aligned linearly in one operative configuration for insertion into an operation site via an aperture configured on the access port 140 and once inserted are articulated about the pitch joints P1 and P2, and moved with the help of the other joints to perform a single port procedure with at least seven axes of movement. Again referring to FIG. 19, the visioning system 170 with an image capturing device 180, particularly, a camera secured to the distal end thereof is inserted into the inside the abdominal cavity 130 inside the patient's body “B”. The pair of articulating arms 110, 120 holds operating tools, such as dissector at the distal end thereof for performing operation on an organ “O” disposed inside the abdominal cavity 130.

The elongate support element 112 and the arm elements 114, 116 configuring the articulating arm 110 are moved and the end effectors 118 disposed at the distal end of the articulating arm 110 rolls, yaws and swivels to achieve movement along at least seven axes of movement to facilitate performing of a surgical procedure. More specifically, referring to FIG. 20a, the elongate support element 112 of the articulating arm 110 has such configuration that enables defining extended and retracted configuration of the articulating arm, thereby defining movement of the articulating arm 110 about first axis “A1” referred to as first linear axis of movement. Referring to FIG. 20a, the first arm element 114 is connected to the elongate support element 112 via the second pitch joint “P2”, thereby facilitating articulation of the first arm element 114 with respect to the elongate support element 112 and defining movement of the articulating arm 110 about second axis “A2” to define a first pitch motion. The element 116 is connected to the element 114 via the first pitch joint “P1”, thereby facilitating articulation of the second arm element 116 with respect to the first arm element 114 and defining movement of the articulating arm 110 about third axis “A3” to define a second pitch motion. Referring to FIG. 20b, the end effectors 118 fitted at the distal end of the second arm element 116 of the articulating arm 110 via the wrist joint “W” rolls either in linear and non-linear configuration of the articulating arm 110 to define movement of the articulating arm 110 about fourth axis “A4” to define wrist roll motion. Further, the end effectors 118 fitted at the distal end of the second arm element 116 via the wrist joint “W” yaws about the wrist joint to define movement of the articulating arm 110 about fifth axis “A5” to define wrist yaw motion. Again referring to FIG. 20a, the elongate support element 112 can swivel sideways thereby defining movement of the articulating arm 110 about sixth axis “A6”. Further referring to FIG. 20b, the elongate support element 112 of the articulating arm 110 swivels to define the movement of the articulating arm 110 about seventh axis “A7”, particularly, the first arm element 112 of the articulating arm 110 swivels by about +/−10 degrees with respect to port entry point to create volumetric work envelop. The elongate support element 112 can be angularly displaced with respect a support structure by an angle between −30 degrees and +30 degrees, the first arm element 114 can be angularly displaced with respect to the elongate support element 112 by an angle between −60 degrees and +60 degrees and the second arm element 116 can be angularly displaced with respect to the first element 114 by an angle between −60 degrees and +60 degrees. With such configuration of the articulating arm, the axis of movements the base link remains fairly stationary other than 10 degree of lateral movement, which significantly reduces outside movements of the articulating arm 110 outside the patient's body above the access port 140 mounted on the incision 150 formed on the patient's body “B”. The end effectors 118 can swivel independently with respect to each other about the hinge joint to provide either or gripping and cutting force to the end effectors. With such configuration the articulating arm 110 achieves movement along at least seven axes of movement, thereby providing maneuverability, proper approach to an operative space, proper articulation and triangulation of forces and enabling a surgeon to perform complex procedures even while operating through a miniaturized single port while still maintaining ergonomic posture and reducing operation time and fatigue.

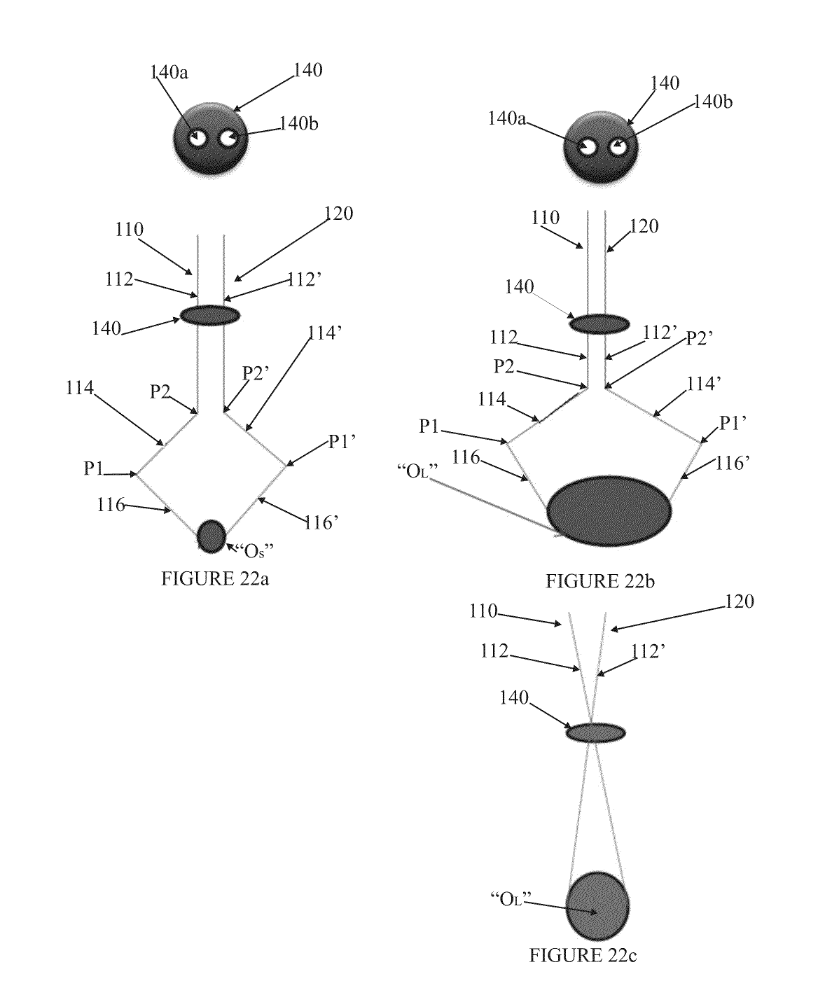

A robotic surgical instrument system for performing surgical procedures is disclosed in accordance with another embodiment of the present disclosure. The robotic surgical instrument system includes a pair of articulating arms 110 and 120, a control and a resilient access port 140. The control independently controls the movements of each of the articulating arms 110 and 120. More specifically, the control facilitates articulation of the arm elements 114 and 116 with respect to each other and the elongate support element 112 of the arm 110 and articulation of the arm elements 114′ and 116′ with respect to each other and the elongate support element 112′ of the arm 120 to enable the articulating arms 110 and 120 to approach and access an organ inside the operation site from opposite sides and achieve triangulation of forces to perform operation at regions, the tissues, objects and organs while still requiring proportionately less actuation forces to generate a predetermined horizontal pull force. The control also facilitates movement of support structure with respect to elongate support element to enable swiveling of the elongate support element. The control further facilitates yawing, rolling and swiveling movements of the end effectors to enable the articulating arms 110, 120 to perform surgical procedure at the operation site after the articulating arms 110, 120 has been introduced in the operation site. The resilient access port 140 has at least three apertures and is mounted on an incision on a patient's body at the operation site, wherein a pair of apertures 140a and 140b (illustrated in FIG. 22a and FIG. 22b) of the at least three apertures receives the pair of articulating arms 110 and 120 for performing a single port procedure on a site corresponding to the incision. In accordance with another embodiment, the access port 140 has two apertures 140a and 140b (illustrated in FIGS. 22a and 22b) for entry of the articulating surgical arms 110, 120, an aperture (not illustrated) for entry of the vision system 170 and another aperture 190 (not illustrated) for introducing a suction pipe inside the operation site for evacuation of tissues from operation site. The number of apertures configured on the resilient access port 140 may vary depending upon requirements. Further, the configuration and the placement of the apertures configured on the resilient access port 140 may vary depending upon requirement.

The robotic surgical instrument system further includes at least one input device, the visioning system 170 and at least one output device. The least one input device co-operates with the control to remotely manipulate the articulating arms 110 and 120 by controlling each of the movements of the arm elements 112 and 114 of the articulating arm 110 and the arm elements 112′ and 114′ of the articulating arm 120 and yawing, rolling and swiveling movements of the end effectors of the articulating arms. Typically, the at least one input device is selected from a group consisting of a joystick, a touch-screen and a foot control pedal. The visioning system has a camera element 180 that is introduced through an aperture of the access port 140 for visioning the operation site. The at least one output device displays images captured by the visioning system 170.

A method for performing a robotic surgery is disclosed in accordance with an embodiment of the present disclosure. The method includes the steps of linearly introducing a pair of articulating arms 110 and 120, via apertures 140a and 140b of a resilient access port 140, into an operation site where a surgery is required to be performed, wherein each articulating arm includes a first arm element 114 and a second arm element 116 of at least a pair of arm elements joined to each other by at least a first pitch joint P1, an elongate support element 112 joined to the first arm element 114 by at least a second pitch joint P2 and a pair of end effectors 118 connected to each other by a hinge joint and to the distal end of the second arm element 116 via a wrist joint W, thereafter, articulating the arm elements 112 and 114 of the articulating arm 110 and the arm elements 112′ and 114′ of the articulating arm 120 in a controlled manner, with respect to each other to approach and access regions, organs, tissues and objects inside the operation site and achieve triangulation of forces to perform operation at the region, organs, tissues and objects while still requiring proportionately less actuation forces to generate a predetermined horizontal pull force and rolling, yawing and swiveling of the end effectors 118 and 118′ to facilitate performing of a single port procedure. The method for performing a robotic surgery also includes the step of controllably skewing the elongate support elements 112 and 112′ corresponding to each of the arms 110 and 120 respectively to relatively displace the arm elements of the respective arms 110 and 120 away from each other. The method further includes the step of controlling the movements of the arm elements of the respective arms 110 and 120 to achieve triangulation of forces at the end effectors 118 and 118′ to perform operation at regions, organs, tissues and objects, and generate a horizontal pull force. Still further, the method includes the step of displacing the support elements 112 and 112′ in an operative configuration between a first arrangement wherein the support elements 112 and 112′ are parallel to each other, and a second arrangement wherein the support elements 112 and 112′ are skewed with respect to each other, wherein in the second arrangement the support elements 112 and 112′ can be configured to be inclined away from each other or inclined crossing to each other along different planes for configuring a criss-cross configuration of the elongate support element 112 and 112′ with pivot at the access port 140. With such configuration the elongate support element 112 and 112′ exert very less forces at the access port 140 and as such very less forces on the portion of the body on which the access port 140 is mounted. Further, pivoting of the elongate support element 112 and 112′ at the access port enables displacing of the arm elements further away from each other inside the patient's body.

The articulating arm 110 configured by joining the elongate support element 112 to the first arm element 114 by the pitch joint P2, and joining the elements 114, 116 by pitch joint P1 and the connecting the end effectors 118 to the distal end of the second arm element 116 is so configured that the elongate support element 112, the arm elements 114, 116, the pitch joints P1 and P2 and the end effectors 118 are arranged linearly in one operative configuration and are linearly introduced into an operation site through one of the plurality apertures configured on the access port 140 mounted on the incision 150 on the patient's body “B” at the operation site, once inserted inside the patient's body “B”, the arm elements 114 and 116 move with respect to each other about the pitch joint P1 and with respect to the elongate support element 112 about the pitch joint P2 to configure a second operative configuration thereof, thereby enabling the articulating arm to approach and access organs, tissues and objects inside the operation site and achieving triangulation of forces to perform operation at regions, the organs, tissues and objects while still requiring proportionately less actuation forces to generate a predetermined horizontal pull force. The end effectors 118 rolls, yaws and swivels to facilitate performing of a surgical procedure.

The articulating arm 120 configured by joining the elongate support element 112′ to the arm element 114′ by pitch joint P2′ and elements 114′ and 116′ to each other by pitch joint P1′ and the connecting the end effectors 118′ to the distal end of the arm element 116′ via a wrist joint ‘W’ is so configured that the elongate support element 112′ and the arm elements 114′, 116′, the pitch joints P1′ and P2′ and the end effectors 118′ are arranged linearly in one operative configuration and are linearly introduced into an operation site through one of the plurality apertures configured on the access port 140 mounted on the incision 150 on the patient's body “B” at the operation site, once inside the patient's body “B”, the arm elements 114′ and 116′ moves with respect to each other about pitch joint P1 and the arm element 114′ moves with respect to the elongate support element 112′ about the pitch joint P2′ to configure a second operative configuration thereof, thereby enabling the articulating arm 120 to approach and access tissues, objects and organs “O” inside the operation site and achieving triangulation of forces to perform operation at regions, the tissues, objects and organs “O” while still requiring proportionately less actuation forces to generate a predetermined horizontal pull force. The end effectors 118′ rolls, yaws and swivels to facilitate performing of a surgical procedure. The articulating arm 120 is structurally and functionally similar to the articulating arm 110 and for the sake of brevity of the present document is not described in detail.

FIG. 22a illustrates a schematic representation depicting a front view of the pair of articulating arms 110, 120 entering an abdominal cavity via the single port 140 having two apertures 140a and 140b (illustrated in top view and also referred to as the first aperture 140a and a second aperture 140b) mounted on the incision formed on the patient's body, wherein the articulating arms 110, 120 are inserted straight into the abdominal cavity, thereafter are moved inside the abdominal cavity to simultaneously approach both sides of a comparatively smaller organ “Os”, tissues and objects. FIG. 22b illustrates a schematic representation depicting a front view of the pair of articulating arms 110, 120 entering the abdominal cavity via the single port 140 having two apertures 140a and 140b (illustrated in top view) mounted on an incision formed on a patient's body, wherein the articulating arms 110, 120 are inserted straight into the abdominal cavity, thereafter are moved inside the abdominal cavity to simultaneously approach both sides of a comparatively larger organ “O_{L}”, tissues and objects if the organ orientation is in different plane, the arms can be selectively skewed to simultaneously approach both sides of a comparatively larger organ “O_{L}”, tissues and objects. Accordingly, such a configuration of the articulating arms 110, 120 of the robotic surgical instrument system enables the articulating arms 110, 120 to operate in pairs and be moved inside the operation site to ensure approach to both large and small sized organs “O_{L}” and “Os” from opposite sides, thereby enabling the articulating arms 110,120 to simultaneously approach both sides of both large and small sized organs “O_{L}” and “Os”, tissues and objects as illustrated in Figures FIG. 22b and FIG. 22a respectively without requiring change in configuration of arms, ports or retraction and reinsertion.

More specifically, the elongate support elements 112 and 112′ are selectively skewed to be in inclined configuration thereof. The support elements 112 and 112′ are displaceable in an operative configuration thereof between a first arrangement wherein the support elements 112 and 112′ are parallel to each other, and a second arrangement wherein the support elements 112 and 112′ are skewed with respect to each other, wherein in the second arrangement the support elements 112 and 112′ can be configured to be inclined away from each other or inclined crossing to each other to configure criss-cross configuration of the elongate support elements 112 and 112′. The articulating arms 110 and 120 are inserted into the operation site in a first configuration in which the corresponding elongate support elements 112 and 112′ are disposed parallel to each other. FIG. 22b illustrates a front view of the pair of articulating arms 110, 120, wherein the corresponding elongate support elements 112 and 112′ are skewed and articulated as required to enable the articulating arms 110 and 120 to simultaneously approach both sides of a comparatively larger organ “O_{L}”, tissues and objects. FIG. 22c illustrates a side view of the pair of articulating arms 110, 120, wherein the corresponding elongate support elements 112 and 112′ are skewed along different planes to be inclined and enable the articulating arms 110 and 120 to simultaneously approach both sides of a comparatively larger organ “O_{L}”, tissues and objects. Further, since the articulating arms 110,120 are independent of each other, only one articulating arm is required to be retracted as opposed to retracting the entire bundle from the patient's body in case of tool change or functionality (grasper etc.) change in the conventional arrangement. The elongate support element 112 and the elongate support element 112′, the arm elements 114, 116 and the arm elements 114′, 116′, the pitch joints P1, P2 and pitch joints P1′, P2′ and the end effectors 118 and the end effectors 118′ corresponding to the articulate arms 110 and 120 respectively are arranged linearly in one operative configuration thereof and are inserted into an operation site through the apertures 140a and 140b respectively configured on the access port 140 mounted on an incision on a patient's body at the operation site in a first configuration and a second configuration. In the first configuration the elongate support elements 112 and 112′ are disposed along parallel planes. In the second configuration the elongate support elements 112 and 112′ are disposed along intersecting planes for configuring criss-cross configuration of the elongate support elements 112 and 112′. More specifically, the access port 140 and the apertures 140a and 140b configured on the access port 140 through which the elongate support elements 112 and 112′ passes are of such configuration so as to facilitate skewing of the corresponding elongate support elements 112 and 112′ so that the elongate support elements 112 and 112′ are in inclined configuration. Such configuration enables the set of arm elements 114, 116 to move relatively away from the set of arms elements 114′, 116′ and with such configuration the articulating arms 110 and 120 articulate while still comparatively farther away from each other to enable the arms to approach comparatively large organs. With such configuration, the sets of arm elements 114, 116 and 114′, 116′ start articulating with respect to each other while still farther from each other, thereby enabling them in approaching comparatively larger organs. The inclination of the elongate support elements 112 and 112′ can be varied based on the size of the organ to be operated. Further, such configuration permits quick and convenient switching of the elongate support elements 112 and 112′ from first configuration to the second configuration. The elongate support elements 112 and 112′ can be skewed to be inclined to approach a wide range of organs, tissues and regions. Further, the elongate support elements 112 and 112′ can be skewed to be inclined along any plane to enable the articulating arms 110 and 120 to perform operation at a wide range of size of organs in different orientation. More specifically, the access port 140 is resilient to facilitate skewing of the elongate support elements 112 and 112′, to change inclination of the elongate support elements 112 and 112′ along parallel planes, thereby facilitating switching between the first configuration and the second configuration. Particularly, the walls of the apertures configured on the access port 140 are resilient to facilitate skewing of the elongate support elements 112 and 112′. Further, with such configuration of the articulating arms 110, 120, the articulating arms 110, 120 can operate on smaller and larger organ without any changes or retraction and reinsertion of the articulating arms 110, 120.

The present invention may be embodied in other specific forms without departing from its spirit or essential characteristics. The described embodiment is to be considered in all respects only as illustrative, and not restrictive. The scope of the invention is, therefore, indicated by the appended claims, rather than by the foregoing description. All changes which come within the meaning and range of equivalency of the claims are to be embraced within their scope.

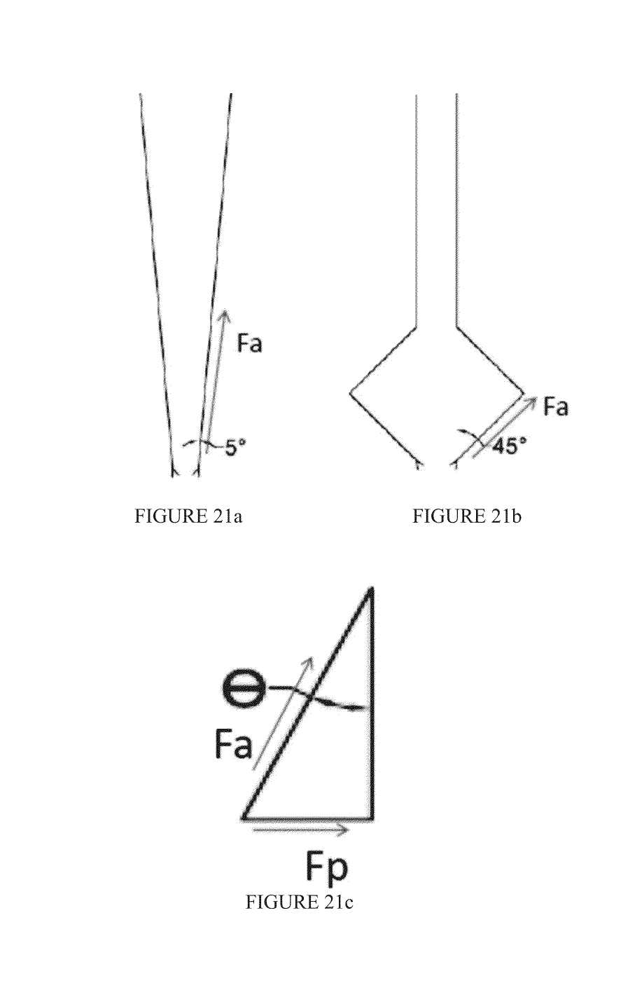

FIG. 21a and FIG. 21b illustrates a schematic representation of a conventional surgical arm and an articulation arm in accordance with an embodiment of the present disclosure, wherein the conventional surgical arm has an angle of approach in the range of 5-10° whereas in case of the articulation arm 110, the articulating elements move with respect to each other and has an angle of approach in the range of 30-60°. FIG. 21c illustrates a force triangulation diagram depicting relation between horizontal pull force Fₚ and pull component along the arm Fₐ as a function of angle of approach θ. In case of the articulation arm 110, the angle of approach is in the range of 30-60°. With such configuration of the articulation arm 110, the articulation arm 110 requires significantly reduced actuation force to achieve the same horizontal pull force at the end effectors disposed at the distal end of the articulating arm 110. More specifically, in case of the conventional arms assuming the angle of approach θ to be in the range of 5°-10°, to achieve 10 N of horizontal pull force Fp, the pull component along arm Fₐ is between 114 N and 57 N. The pull component along arm Fₐ is calculated using the formula Fₚ=Fₐ/Sin θ, wherein Fₐ is the pull component along the arm, θ is the angle of approach and Fₚ is the horizontal pull force. In case of the articulating arm of the present disclosure assuming the angle of approach θ to be in the range of 30-60°, to achieve 10 N of horizontal pull force Fp, the pull component along arm Fₐ is about 14 N. Accordingly, from the above example it is clear that the articulating arm of the present disclosure requires significantly reduced force to achieve the same horizontal pull force at the end effectors disposed at the distal end of the articulating arms. The articulating arm for a robotic surgical instrument system can be moved inside an operation site to achieve triangulation of forces that result in significant reduction in the actuation forces, preferably less than 30 N, required to generate for example about 10 N horizontal pull force at the end effectors, such configuration enables the articulating arms to apply proportionately maximum counteracting forces to organs at the operation site.

In order to generate horizontal pull force at the distal end an actuation force is required at the proximal end. Due to the configuration of the articulation arm of the present disclosure, the actuation force required is significantly less compared to conventional surgical arms. For example, to generate for example 10 N of horizontal pull force at the distal end the actuation force required in the disclosed surgical arm is in the range of 11 N-20 N (assuming angle of approach θ=30°-60°) while for the conventional surgical arms the actuation force required is in the range of 114 N-57 N (assuming θ=5°-10°—refer page 2). With lower actuation force requirement, miniature components could be used for actuation and transmission of the force resulting in smaller size of the arm. Accordingly, the articulation arm of the present disclosure is having compact configuration as compared to the conventionally known surgical arms.

### TECHNICAL ADVANCEMENTS

The technical advancements of the present disclosure include:
a robotic surgical instrument system having arms that are versatile and allow for adjustment according to size of regions, organs, tissues and objects to be approached inside the operation site;
a robotic surgical instrument system having arms that can approach and perform operation on regions, organs, tissues and objects of varying sizes;
a robotic surgical instrument system having a plurality of articulating arms that facilitates minimal invasive surgery, thereby avoiding larger cuts and ensuring lesser trauma to patient, less post-operative pain and faster recovery of the patient;
a robotic surgical instrument system having a plurality of articulating arms that facilitates single port surgery and therefore ensures various benefits of single port surgery that includes better cosmetic results for patients, less blood loss and easy tissue retrieval;
a robotic surgical instrument system having a plurality of articulating arms that maintains benefits of the single port surgery while still reducing surgeon's fatigue that is prevalent in the case of manual single port surgery;
an articulating arm for a robotic surgical instrument system that achieves movement along at least seven axes of movement, thereby enabling a surgeon to perform complex procedures even while operating through a miniaturized single port;
an articulating arm for a robotic surgical instrument system that can be used together along with another arm to define dual arm configuration of a robotic surgical instrument system and enabling the arms to approach and access an organ inside an operation site from opposite sides, thereby enabling the arms to simultaneously approach both sides of a larger organ or objects like tumors and facilitating better control during a surgical procedure;
an articulating arm for a robotic surgical instrument system that can be moved inside an operation site to achieve triangulation of forces, thereby enabling the articulating arms to apply proportionately maximum counteracting forces to organs at the operation site;
an articulating arm for a robotic surgical instrument system that has such configuration that enables the articulating arm to be inserted straight inside an operation site though a miniature aperture configured on a single access port mounted on an incision on a patient's body at the operation site and to be moved inside the operation site to ensure access to organs in the operation site;
an articulating arm for a robotic surgical instrument system that permits single port entry thereof into an operative space inside a patient's body, thereby requiring minimum incision for carrying a surgical procedure;
an articulating arm for a robotic surgical instrument system that can be moved remotely, thereby enabling the articulating arm to be inserted straight into an abdominal cavity, thereafter be moved inside the abdominal cavity;
a robotic surgical instrument system having a plurality of articulating arms, wherein robotic arms thereof can be conveniently and remotely controlled using joysticks and as such ensures ergonomic posture for the surgeon, thereby reduces surgeon's fatigue and chances of error due to fatigue; and
an articulating arm for a robotic surgical instrument system, wherein arm elements configuring the articulating arm are connected to each other by pitch joints, thereby facilitating triangulation, enhancing leverage and providing actuating forces at the distal end of the articulation arm to enhance maneuverability of the articulation arm without requiring a lot of movements outside a patient's body above an access port mounted on an incision formed on the patient's body, wherein such movements outside the patient's body can be uncomfortable and unsafe for the surgeon and the people working in close proximity of patient;
an articulating arm for a robotic surgical instrument system that reduces exposure of internal organs to possible external contaminants thereby reducing risks of acquiring infections;
an articulating arm for a robotic surgical instrument system such that the organs could be approached just like multiport surgery but through single port, thereby reducing the learning curve of the surgeons;
an articulating arm for a robotic surgical instrument system that does not exert any lateral forces on an access port mounted on an incision formed on the patient's body and the patient's abdomen wall;
an articulating arm for a robotic surgical instrument system that does not require movement outside a patient's body other than swivel of about 10 degrees for articulating the arms;
arms for a robotic surgical instrument system that are independent of each other, thereby facilitating convenient tool change or functionality (grasper etc.) change as only one arm is required to be retracted as opposed to retracting the entire bundle from the patient's body in case of conventional robotic surgical instrument system; and
an articulating arm for a robotic surgical instrument system that has such configuration that the roll, pitch and finger movement are provided at distal end, thereby enabling control of the articulating arm from proximal end.

Throughout this specification the word “comprise”, or variations such as “comprises” or “comprising”, Will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed anywhere before the priority date of this application.

1. An articulating arm for a robotic surgical instrument system for performing single port procedures, said arm comprising:
at least a pair of arm elements, wherein a first arm element and a second arm element of said pair of arm elements are joined to each other by at least one first pitch joint;
an elongate support element connected at one end thereof to said first arm element by at least one second pitch joint and other end of said elongate support element is connected to a support structure and is functionally coupled to a control of said robotic surgical instrument system;
a pair of end effectors connected to each other by means of a hinge joint, wherein said hinge joint facilitates swiveling of said end effectors relative to each other to configure scissoring action of said end effectors;
a wrist joint connecting said pair of end effectors to the distal end of said second arm element, wherein said wrist joint is adapted to facilitate yaw movement and rolling movement of said end effectors,
said elongate support element, said arm elements, said pitch joints and said end effectors adapted to be aligned linearly in one operative configuration for insertion via an aperture of a port into an operation site and once inserted adapted to be articulated about said pitch joints, and moved with the help of the other joints to perform a single port procedure with at least seven axes of movement.

2. The articulating arm as claimed in claim 1, wherein each pitch joint comprises:
a pair of pitch base members extending from said first arm element, wherein said pitch base members are spaced from each other; and
an axis member pivotably supported between said pitch base members and adapted to support thereon said second arm element.

3. The articulating arm as claimed in claim 2, wherein said axis member is a yaw axis member.

4. The articulating arm as claimed in claim 1, wherein in a first operative configuration said arm elements, said pitch joints and said end effectors are adapted to be linearly aligned and move along a linear axis to define movement of the articulating arm about a first linear axis of movement, wherein in a second operative configuration said first arm element is adapted to move with respect to said elongate support element to define movement of the articulating arm about a second axis to define a first pitch motion; wherein in a third operative configuration said second arm element and said first arm element are adapted to move with respect to each other to define movement of the articulating arm about a third axis to define a second pitch motion, wherein in a fourth operative configuration said end effectors fitted to the distal end of said second arm element via said wrist joint is adapted to roll either in linear and non-linear configuration of said articulating arm to define movement of the articulating arm about a fourth axis to define wrist roll motion, wherein in a fifth operative configuration said end effectors fitted to the distal end of said second arm element via said wrist joint is adapted to be yaw about said wrist joint to define movement of said articulating arm about a fifth axis to define wrist yaw motion, wherein in a sixth operative configuration the elongate support element of said articulating arm is adapted to swivel sideways defining movement of the articulating arm about a sixth axis of movement, wherein in a seventh operative configuration said elongate support element of the articulating arm is adapted to swivel by +/−10 degrees with respect to a port entry point to create volumetric work envelop and define the movement of said articulating arm about a seventh axis, wherein in an eighth operative configuration said end effectors are adapted to swivel independently with respect to each other about the hinge joint to provide either of gripping and cutting force to said end effectors.

5. A robotic surgical instrument system for performing surgical procedures comprising:
a pair of articulating arms as claimed in claim 1;
a control adapted to independently control the movements of each of said articulating arms; and
a resilient access port having at least three apertures adapted to be mounted on an incision on a patient's body at said operation site, wherein a pair of apertures of the at least three apertures adapted to receive the pair of articulating arms for performing a single port procedure on a site corresponding to said incision.

6. The robotic surgical instrument system as claimed in claim 5, further comprising:
at least one input device cooperating with said control to remotely manipulate said articulating arms by controlling each of the movements of said arm elements of said articulating arms and yawing, rolling and swiveling movements of said end effectors of said articulating arms;
a visioning system having a camera element adapted to be introduced through an aperture of said port for visioning said operation site; and
at least one output device adapted to display images captured by said visioning system.

7. The robotic surgical instrument system as claimed in claim 6, wherein said at least one input device is selected from a group consisting of a joystick, a touch-screen and a foot control pedal.

8. A method for performing a robotic surgery comprising the steps of:
linearly introducing, via apertures of a resilient access port, into an operation site where a surgery is required to be performed, a pair of articulating arms, wherein each articulating arm comprises a first arm element and a second arm element of at least a pair of arm elements joined to each other by at least a first pitch joint, an elongate support element joined to said first arm element by at least a second pitch joint and a pair of end effectors connected to each other by a hinge joint and to the distal end of said second arm element via a wrist joint;
articulating said arm elements in a controlled manner, with respect to each other to approach and access regions, organs, tissues and objects inside said operation site and achieve triangulation of forces to perform operation at regions, said organs, tissues and objects while still requiring proportionately less actuation forces to generate a predetermined horizontal pull force; and
rolling, yawing and swiveling of said end effectors to facilitate performing of a single port procedure.

9. The method as claimed in claim 8, which includes the step of controllably skewing said elongate support elements corresponding to each of said arms to relatively displace the arm elements away from each other.

10. The method as claimed in claim 8, which includes the step of controlling the movements of the arms to achieve triangulation of forces at the end effectors to perform operation at regions, organs, tissues and objects, and generate a horizontal pull force.

11. The method as claimed in claim 8 which includes the step of displacing the support elements in an operative configuration between a first arrangement wherein the support elements are parallel to each other, and a second arrangement wherein the support elements are skewed with respect to each other, wherein in the second arrangement the support elements can be configured to be inclined away from each other or inclined crossing to each other with pivot at said access port.

12. The method as claimed in claim 8, wherein the elongate support element can be angularly displaced with respect a support structure by an angle between −30 degrees and +30 degrees, the first arm element can be angularly displaced with respect to the elongate support element by an angle between −60 degrees and +60 degrees and the second arm element can be angularly displaced with respect to the first element by an angle between −60 degrees and +60 degrees.
